(19) United States
(12) Patent Application Publication
(10) Pub. No.: US 2015/0094802 A1
(43) Pub. Date: Apr. 2, 2015

(54) SINGLE-RING CARDIAC VALVE SUPPORT

(71) Applicant: MVALVE TECHNOLOGIES LTD., Herzliya (IL)

(72) Inventors: Maurice BUCHBINDER, La Jolla, CA (US); Shay DUBI, Tel-Aviv (IL); Amit TUBISHEVITZ, Tel-Aviv (IL); Avi EFTEL, Tel-Aviv (IL)

(21) Appl. No.: 14/471,575

(22) Filed: Aug. 28, 2014

Related U.S. Application Data
(63) Continuation-in-part of application No. PCT/IL2013/000025, filed on Feb. 28, 2013.
(60) Provisional application No. 61/604,083, filed on Feb. 28, 2012.
(60) Provisional application No. 61/620,679, filed on Apr. 5, 2012.
(60) Provisional application No. 61/639,924, filed on Apr. 29, 2012.
(60) Provisional application No. 61/650,559, filed on May 23, 2012.
(60) Provisional application No. 61/752,996, filed on Jan. 16, 2013.
(60) Provisional application No. 61/752,994, filed on Jan. 16, 2013.
(60) Provisional application No. 62/032,581, filed on Aug. 3, 2014.
(60) Provisional application No. 62/039,937, filed on Aug. 21, 2014.

(51) Int. Cl. A61F 2/24

(52) U.S. Cl. CPC A61F 2/2409; A61F 2/2418; A61F 2230/0065; A61F 2220/0016; A61F 2220/0033
USPC 623/2.18; 623/2.38

(57) ABSTRACT

The present invention is primarily directed a prosthetic cardiac valve support device adapted for endovascular delivery to a cardiac valve, comprising a single ring-shaped support element having an inner diameter and an outer diameter, wherein said support element has an outer perimeter that is entirely rigid, wherein said support element is fitted with one or more intra-ventricular and/or intra-atrial stabilizing elements, and wherein said support element has a collapsed delivery configuration and a deployed configuration.

### BACKGROUND OF THE INVENTION

Heart valve regurgitation occurs when the heart leaflets do not completely close when the heart contracts. When the heart contracts, blood flows back through the improperly closed leaflets. For example, mitral valve regurgitation occurs when blood flows back through the mitral valve and into the left atrium when the ventricle contracts.

In some instances regurgitation occurs due to disease of the valve leaflets (e.g., primary, or “organic” regurgitation). Regurgitation can also be caused by dilatation of the left ventricle, which can lead to secondary dilatation of the mitral valve annulus. Dilation of the annulus spreads the mitral valve leaflets apart and creates poor tip coaptation and secondary leakage, or so-called “functional regurgitation.”

Currently, primary regurgitation is corrected by attempting to remodel the native leaflets, such as with clips, sutures, hooks, etc., to allow them to close completely when the heart contracts. When the disease is too far advanced, the entire valve needs to be replaced with a prosthesis, either mechanical or biologic. Examples include suture annuloplasty rings all the way to actual valve replacement with leaflets, wherein the suture rings are sutured to the mitral valve annulus. Annuloplasty rings, which are also sutured to the annulus, have also been used to attempt to remodel the annulus, bringing the native leaflets closer together to allow them to properly close.

Based on the success of catheter-based aortic valve replacement there is growing interest in evaluating similar technologies to replace the mitral valve non-invasively using similar types of replacement valves.

Unlike the aortic valve, however, the mitral valve annulus does not provide a good landmark for positioning a replacement mitral valve. In patients needing a replacement aortic valve, the height and width of the aortic annulus are generally increased in the presence of degenerative disease associated with calcium formation. These changes in tissue make it easier to properly secure a replacement aortic valve in place due to the reduced cross-sectional area of the aortic annulus. The degenerative changes typically found in aortic valves are not, however, present in mitral valves experiencing regurgitation, and a mitral valve annulus is therefore generally thinner than the annulus of a diseased aortic valve. The thinner mitral valve annulus makes it relatively more difficult to properly seat a replacement mitral valve in the native mitral valve annulus. The general anatomy of the mitral valve annulus also makes it more difficult to properly anchor a replacement mitral valve in place. The mitral valve annulus provides for a smoother transition from the left atrium to the left ventricle than the transition that the aortic valve annulus provides from the aorta to the left ventricle. The aortic annulus is anatomically more pronounced, providing a larger “bump” to which a replacement aortic valve can more easily be secured in place.

In general, the aortic valve annulus is smaller than the mitral valve annulus. It has been estimated that the mitral valve annulus is about 2.4 cm to about 5 cm in diameter, while the aortic valve annulus has been estimated to be about 1.6 cm to about 2.5 cm in diameter.

The larger mitral valve annulus makes it difficult to securely implant current percutaneously delivered valves in the native mitral position. Current replacement aortic valves are limited in the amount of radial expansion they can undergo during deployment and implantation. To provide a replacement aortic valve that has an expanded configuration such that it can be securely anchored in a mitral valve annulus would require that the collapsed delivery profile of the replacement aortic valve be increased. Increasing the collapsed delivery profile, however, would make endovascular delivery more dangerous for the patient and more difficult to navigate the vasculature with a larger diameter delivery system.

Some attempts have been made to deliver and implant a one-piece replacement mitral valve, but it is difficult to provide a device that can be collapsed down to have a sufficiently small delivery profile and still be able to be expanded and secured in place within the mitral valve via a vascular access site.

A two-ring valve-support device suitable for endoscopic delivery was disclosed in a co-owned, co-pending US application (Ser. No. 13/224,124, filed on Sep. 1, 2011 and published as US 2012/0059458). While this device provided technical solutions to most of the problems associated with prior art approaches (as described hereinabove), it was felt that in certain clinical situations it would be advantageous to be able to implant a valve-support which would be at least as stable as the above-mentioned two-ring support following implantation and which would also provide equal or superior support for a subsequently-implanted replacement cardiac valve, but which would be simpler in construction, have a smaller crossing profile and occupy less space within the heart cavity following implantation.

### SUMMARY OF THE INVENTION

The present invention is primarily directed to a prosthetic cardiac valve-support device comprising a single valve-support element (also referred to hereinbelow as the “support element”) which is suitable for endovascular delivery to the region of an anatomical cardiac valve, wherein said support element is generally provided in the form of a single annular-shaped ring having an inner diameter and an outer diameter, said inner diameter defining the external boundary of an internal space. Based on a consideration of the anatomical features of cardiac valves (particularly the mitral valve), an annular-shaped simplified, single-ring valve support device implanted on one side (preferably the superior side) of the annulus, or within the annulus itself would not intuitively be considered to be capable of being retained in position over a long period of time. However, it was unexpectedly found by the present inventors that said device was itself mechanically stable following implantation, and furthermore, was capable of providing a stable base for the subsequent implantation of a replacement valve (preferably a replacement aortic valve) within the central space of said annular support device.

Thus, in one aspect, the present invention provides a valve-support device comprising a single ring-shaped annular support element, wherein said support element has a collapsed delivery configuration and a deployed configuration. In one embodiment, the support element is provided in the form of flat annular ring, preferably constructed from a material having superelastic and/or shape memory properties. One example of such a suitable material is Nitinol, which possesses both of the aforementioned physical properties. These properties may be utilized in order to permit said device, following its delivery in a collapsed conformation, to return to an expanded memory configuration after being heated above its transition temperature. In the radial plane (i.e. the plane in which the native cardiac valve leaflets are disposed when in their closed position), the size of the annular support element may be defined in terms of its outer radius (Ro), its inner radius (Ri) and the difference between these two radii (Rd). It should be appreciated that Ro is determined by the diameter of the mitral valve annulus into which the valve support device will be implanted. Ri, however, is determined by the outer diameter of the replacement heart valve that will be inserted into the central space of the support device. Generally, the prosthetic aortic valves used in conjunction with the valve support device of the present invention have an external diameter considerably less than that of the mitral valve annulus. It may therefore be appreciated that Rd approximately corresponds to the annular gap between the small outside-diameter replacement valve and the relatively large diameter mitral valve annulus. Preferably, Rd is in the range of 1-14 mm. In most embodiments of the valve support device of the present invention, the inner radius of the single-ring support element is in the range of 23-29 mm and the outer radius thereof is in the range of 30-50 mm.

With regard to the thickness of the support element (t) (as measured along the longitudinal axis of the element when in situ), t represents a compromise between the need for minimizing this parameter in order to facilitate crimping and insertion into a delivery catheter, and the need for the support device to be sufficiently rigid such that it is able to withstand the forces exerted by the beating heart without buckling. In one typical, non-limiting example, t is 0.4 mm, while Rd has a value of 5.5 mm. Indeed, as a general rule, in most embodiments of the annular support element of the present invention, Rd is significantly larger than t. For example, in many cases Rd may be between 2.5 and 35 times larger than t, more preferably between 10 and 20 times larger than t. It may be appreciated from the foregoing explanation that the ratio between Rd and t has functional significance for the valve support device of the present invention.

As indicated hereinabove, in a preferred embodiment of the invention, the valve support device is used to assist in the implantation of a prosthetic aortic valve into the mitral valve annulus of a human subject in need of such implantation. The thickness of the support device is generally in the range of 0.25-0.6 mm, more preferably 0.4 mm.

The annular support element may have an outline shape that is circular, elliptical or any other form that permits it to be adapted to make close contact with the inner cardiac wall upon implantation in the region of a cardiac valve annulus.

One particular feature of the valve support device of the present invention is the fact that the outer perimeter of the annular support structure is entirely rigid, such that in its deployed configuration, it is not possible to cause further expansion of the outer diameter of said device.

As mentioned hereinabove, unlike in the case of the aortic valve, the pathologically-involved mitral valve is generally not associated with increased calcification. One consequence of this lack of calcification is that it is not possible to increase stabilization of single-ring valve support devices within the mitral valve annulus by means of elements that exert their stabilizing forces in a radial direction. The reason for this is that he soft tissue of the uncalcified annulus in this situation would simply react to the applied radial forces by expanding in a radially-outward direction, thereby tending to reduce the contact between the support device and the tissue. Thus, in the case of the mitral valve, additional stabilization, if required, can only be achieved by means of stabilizing elements that apply forces on the heart tissues along the longitudinal axis. In this regard, it is to be noted that for the purposes of the present disclosure, the term ‘radial’ refers to the plane of the anatomical valve when the native leaflets are closed. The term ‘longitudinal’ refers to a direction that is at 90 degrees to the radial direction, namely approximately parallel to an imaginary line drawn from the cardiac apex to the cardiac base.

Thus, in preferred embodiments of the invention, the mechanical stability of the single-ring support element is enhanced by the use of stabilizing elements, wherein said stabilizing elements are adapted to apply stabilizing forces to the heart tissue in the longitudinal direction, and/or heart tissue anchoring means.

Thus, in some embodiments, the support element is fitted with heart tissue anchoring means adapted to securely anchor said support element to the heart wall. Non-limiting examples of such anchoring means include hooks and spirals.

In some embodiments, the cardiac valve support further comprises one or more stabilizing elements, the function of which is to provide additional stabilization of said support within the ventricle and/or atrium. Preferably, the cardiac valve support is fitted with two or more stabilizing elements, more preferably two such elements attached to the support element such that the angular separation between them (measured around the circumference of the annular support element is approximately 180 degrees (+/20 degrees). This particular arrangement ensures that in use, the stabilizing elements can be positioned in the region of the medial and lateral mitral valve commissures. The advantage of such an arrangement is that the alignment of the support device stabilizing elements along the line of the native commissure ensures that said stabilizing elements will not interfere with the functioning of the native valve during the period prior to the deployment of a replacement valve within the central space of said support device.

The aforementioned stabilizing elements may be provided in any suitable form, including (but not limited to) additional complete ring structures, partial rings, curved arms or wings, elongate arms or wings and levered arms or wings. Examples of each of these types of stabilizing element are provided hereinbelow.

Thus, in some embodiments, the cardiac valve support comprises one or more intra-ventricular stabilizing elements, one or more intra-atrial stabilizing elements. In other embodiments, the cardiac valve support will be fitted with at least one intra-ventricular stabilizing element and at least one intra-atrial stabilizing element. Although in some cases, the stabilizing means include one or more elements that become physically attached to the cardiac tissue (e.g. in the atrial or ventricular walls), in many other embodiments, said stabilizing means provide additional mechanical stability by means of applying generally longitudinally-directed forces on the inner surface cardiac wall without being physically connected to the subsurface cardiac tissues.

In some other preferred embodiments, the stabilizing elements are provided in the form of elongate anchoring wings that are cut out of the same disc that is used to form the annular support ring itself. Said wings are used to anchor and stabilize the support device in its working location, by means of applying pressure to the inner ventricular wall. In most of the preferred embodiments described herein, the valve support devices comprise only two such anchoring wings. However, certain versions of the device may possess more than two wings.

In general, the anchoring wings of this embodiment are longer than the intra-ventricular and intra-atrial stabilizing structures disclosed hereinabove, and described in more detail hereinbelow. In many cases, this increased length of the anchoring wings is advantageous, since it allows said wings to make contact with a larger area of the ventricular wall surface, thereby resulting in improved stabilization of the support device.

In most preferred embodiments of this aspect of the invention, the single ring support structure contains only two wings, spaced apart by 180 degrees+/−a few degrees. The reason for this is that generally, the wings must be aligned along the mitral valve commissure in order to prevent hindrance of the native valve function during implantation of the replacement valve.

However, despite the need for the wings to be disposed opposite each other, most of the wing designs are asymmetric—that is, the two wings are not formed exactly opposite each other (i.e. exactly 180 degree separation), in order to avoid problems during crimping of the device prior to loading it into the delivery catheter. Rather they are arranged side-by-side when the disc is in a flat conformation (before the wings are bent downwards).

In a still further preferred embodiment, the stabilizing elements are provided in the form of lever-operated anchoring means, wherein said anchoring means comprise one or more anchoring arms and an equal number of fulcrums. Said anchoring arms and fulcrums are mutually disposed such that each anchoring arm is capable of being pivoted in a superior-lateral direction about its fulcrum upon application of a radially-outward force to said anchoring arm. Since this pivotable structure acts as a lever, it is thus able to apply significantly greater forces onto the ventricular wall than would be possible if the anchoring arm were constructed as an essentially static structure. In this way, this embodiment of the stabilizing elements of the present invention is capable of applying forces of sufficient magnitude to the ventricle, thereby enabling the single-ring support device to resist the strong displacement forces generated during ventricular systole. However, in order for lever structure of the present invention to function in its intended way, it is necessary to solve two further technical problems: firstly, the need to apply a sufficiently large force to the anchoring arms in order to cause them to pivot laterally and, in turn, exert similarly large forces on the inner ventricular wall, and secondly the need to time the generation of this laterally-directed expansive force such that said force is applied only when needed—that is, during the second stage of the two-stage replacement valve implantation procedure. In order to explain this second point, it is necessary to briefly consider the manner in which the mitral valve normally functions. Thus, during early systole, the intra-ventricular pressure increases to a point such that the forces that are thereby exerted on the mitral valve leaflets are sufficient to cause them to close, thereby preventing retrograde flow of blood from the left ventricle into the left atrium. During the first stage of the two-stage procedure (implantation of the support structure), the support device is not subjected to strong displacing forces during ventricular systole, due to the closure of the native leaflets, resulting in complete (or near-complete) separation between the left ventricle and left atrium. Furthermore, even when the native leaflets are open (during diastole) the valve support device is subjected to only very low pressure since firstly, the surface area of the ring-shaped support device is small, and secondly, most of said surface area is not situated in the path of the fluid flow between the atrium and ventricle. However, during the second stage of the two-stage method (implantation and expansion of the replacement valve), the native mitral valve leaflets become displaced laterally. Said leaflets are, in this way, prevented from closing during early systole. Consequently, during ventricular systole, very strong upwardly-directed forces are exerted on the replacement valve leaflets, thereby causing them to close. Since the replacement valve leaflets now form a single structure, together with the valve support device, the forces acting on said replacement valve leaflets would cause displacement of the attached valve support device were it not strongly anchored to the ventricular wall. Thus, it is during this second stage of the implantation procedure that it is essential that the anchoring arms of the support device are able to strongly engage with the ventricular wall, in order to counter the sudden increases in the displacing forces applied to said device.

The two aforementioned technical problems have been solved in an ingenious manner by the present inventors by means of exploiting the radial expansion of the replacement valve (during the second stage of the two-stage implementation method), either by means of an inflatable balloon or by the use of a self-expandable stent. In this way, the outwardly-radial forces exerted by the balloon (or self-expanding stent) are transferred to the medial portion of each of the anchoring arms in the support device. Said arms are then caused to pivot about their fulcrum (more details concerning which will be provided hereinbelow). This pivoting motion then continues until the lateral portion of each anchoring arm makes contact with the ventricular wall (or, in some embodiments, together with the medial portion, causes pinching of the native valve leaflets, or in other embodiments come in contact with the device lateral attachment wings (shown in FIG. 10—thus increasing the axial force attaching the wings to the left ventricle and increasing the anchoring force of the wings). In summary: the forces applied by the expanding replacement valve cause radial expansion of the anchoring arms. As a result of the lever arrangement of said arms, the magnitude of the radially-directed force generated by the expanding valve is amplified. An additional benefit derived from the pivotable arm arrangement is that the angle formed between the lateral extremity of each expanded anchoring arm and the tissue of the ventricular wall becomes altered, such that said arm transfers said radially-directed force to said tissue in an axial direction (that is, along the longitudinal axis of the free lateral extremity of the anchoring arm). This directional effect is highly advantageous, since the geometry of anchoring arms is such that they are able to apply greater forces on the heart wall in the direction of their longitudinal axis than if said forces were to be applied at 90 degrees to said axis. In this regard, it should be noted that the aforesaid directional effect does not require that it is the free end of the anchoring arms that make contact with the tissue. Indeed, in certain circumstances such an arrangement may prove undesirable since it may result in trauma to the ventricular tissue. Rather, it is sufficient that a short length of the terminal (i.e. lateral-most) portion of the anchoring arm is angled, thereby forming a non-traumatic base. In such an arrangement, most of the forces exerted by the ventricular wall onto the anchoring arms are still directed axially (i.e. in a longitudinal direction), and thus buckling of the said arms is prevented. Finally, the fact that the force-generating step is the expansion of the replacement valve results in the high-magnitude forces being applied by the anchoring arms at exactly the right moment—that is, from the moment when the native valve leaflets have become immobilized. Preferably the anchoring arm is constructed such that it is bent at a point along its length, such that said arm may be considered to comprise a medial portion and a lateral portion, wherein said portions form an angle greater than 0 degrees between them. It is to be noted that this angle may become larger or smaller during the pivoting movement of the anchoring arms. However, in one preferred embodiment, the angle is progressively reduced to almost 0 degrees (i.e. nearly complete closure of the lateral portion on to the medial portion, as the lateral expansion continues towards its endpoint. Further details of this pivotable-arm embodiment will be described hereinbelow.

In some embodiments the support element is fitted with replacement valve engagement means adapted to securely engage a replacement heart valve. In some embodiment, the engagements means can have anchoring and/or locking elements adapted to securely lock with a portion of a replacement heart valve. In other embodiments, the replacement valve engagement means are formed from a soft biocompatible material (such as a biocompatible fabric, silicon, PET etc.) which is fitted to the external surface of portions of the support element. In these embodiments, the soft, compressible nature of the biocompatible material permits certain portions thereof to be compressed by the struts or other structural elements of the replacement valve, upon expansion within the lumen of the valve support. Other portions of the soft biocompatible material which are not compressed by the expanded replacement valve protrude into the internal space of said valve between the struts and/or other structural elements. The protrusions formed in this way engage and grip the replacement valve thereby preventing its movement in relation to the valve support. In other embodiments, the replacement valve engagement means comprise rigid anchors of a size and shape such that they are capable of entering the internal space of the replacement valve between its struts and/or other structural elements, upon expansion of said valve within the internal space of the valve support.

It is to be noted that most of the prosthetic heart valves intended for transcatheter delivery that are in current use are designed to be implanted in a tube-like area within the body—for example within the aorta. Indeed, in many cases, the prosthetic valve itself has a tubular structure. As a result, the implantation of such valves within a ring-like valve support device element may be more difficult, since in the absence of the aforementioned tube-like structure, the prosthetic valve may require more accurate positioning thereby extending the length of the cardiac procedure, and may furthermore lead to excessive wear of the valve.

In order to improve the compatibility of the support element with tubular replacement valves, in some preferred embodiments of the present invention, the annular single-ring support element further comprises one or more height-increasing elements attached to the inner circumference of the annular support ring. The advantages of the height-increasing elements stem from the fact that in essence they transform the flat ring shape into a tube-like shape, thus better mimicking the shape into which the said prosthetic valves were originally designed for (since both the aortic valve and the aorta have an essentially tubular shape). Further details of said height-increasing elements will be provided hereinbelow, with reference to the accompanying drawings.

In some preferred embodiments of the present invention, the inner perimeter of the annular single-ring support element is able to elastically deform in a radial direction. In this regard, it is to be appreciated that the annular shaped elastically-deformable support element of the present invention possesses an external diameter and an internal diameter. In use, a support element of the present invention is selected such that the replacement cardiac valve, in its fully expanded, deployed configuration, has an external diameter which is slightly larger than the inner diameter of said support element when the latter is at rest. Then, upon deployment of the replacement valve within the internal space of the valve support element, the internal diameter of said support element, by virtue of its elastically-deformable nature, is increased. The replacement valve is thus held firmly in place within the support element by means of the radially-inward forces that are exerted by said support element on said valve as a result of the tendency of the elastic inner surface of said support element to return to its rest position.

It is to be noted that the elastically-deformable support element may be constructed such that either its entire inner surface is elastically deformable or, alternatively, it may be constructed such that only certain discrete regions thereof are elastically deformable.

Currently available Aortic valves which may be implanted via a transfemoral approach or a trans-apical approach are generally made either of balloon expandable material (for example Stainless steel—such as Sapien stented valve, Edwards Inc.) or self-expanding material (for example Nitinol—such as the CoreValve stented valve, Medtronic inc., and Lotus stented valve, Sadra, Boston Scientific Inc.).

In balloon expandable valves, after deployment and expansion of the valve in its position the stent of the valve has a recoil phenomenon. This means that immediately after maximal balloon expansion, when the balloon is deflated, there is some recoil, some “closing back” of the stent. This effect is a physical mechanical property of the metallic balloon expandable stent. When implanted in the Aorta, the Aortic wall is elastic, and after its expansion it applies an inward force on the stent, maintaining it in position. However, if one inflates such a stented valve in a completely rigid tube/element than immediately after expansion the stent will have some recoil, but the rigid element will not have any recoil due to its rigidity—hence there will always be some space between the stent and the rigid element, and the stent will not be held in its place by strong forces.

Self-expanding stents do not display recoil phenomena; however they present a different challenge for deployment within a valve-support. The externally directed forces applied by self-expanding stents is lower than that of balloon expandable stents, hence it is a significant challenge to ensure that a self-expanding stented valve will be deployed and secured into a valve-support, without being dislocated during the cardiac cycle. Hence there is a significant advantage if the valve support has one or more support elements which are capable of applying internally directed radial forces, which increase the forces attaching the valve to the valve support and ensure the valve will not be dislocated.

In some further preferred embodiments of the invention, at least one segment of the single-ring annular support element has an external diameter that is larger than the diameter of the cardiac annulus into which said support element will be implanted (hereinafter referred to as “enlarged diameter segments” or similar), and wherein at least one other portion of said annular element has an external diameter that is smaller than the diameter of said annulus (hereinafter referred to as “reduced diameter segments” or similar). It may thus be appreciated that said reduced diameter segments interrupt the otherwise-ring shaped outer circumference of the support element of the present invention.

In one preferred embodiment of the present invention, the support element has two enlarged diameter segments and two reduced diameter segments. In another preferred embodiment the support element comprises four enlarged diameter segments and four reduced diameter segments.

It will be appreciated that, upon implantation of the cardiac valve support element of the present invention into the region of the cardiac annulus, each of the enlarged diameter segments will form a fluid-tight seal against the tissue of the anatomic annulus. Conversely, a small aperture will be created between each of the reduced diameter segments and the adjacent portion of the annulus, thereby permitting the limited peri-valvular flow of blood between the ventricle and atrium on the side of the heart in which said support element is implanted. In this way, the fluid pressure (and hence force) exerted by the contracting heart on the cardiac valve support (and on a replacement valve situated within said support) will be reduced. Additionally, this unique design reduces the afterload against which the ventricle contracts, since it allows a controlled limited regurgitation, and thus may have beneficial clinical effects on ventricular function, reducing ventricular wall stress and oxygen consumption.

In some embodiments the support element has at least one coupling element adapted to reversibly couple to a delivery system. The at least one coupling element can be a threaded bore.

In a further preferred embodiment of the present invention, the annular support element is fitted with at least one wing-like drape element made of a biocompatible fabric. The drape functions as a sealing element, positioned between the support element and the mitral annulus, thereby preventing paravalvular leakage (PVL) after implantation of the valve support in the mitral annulus.

In one preferred embodiment of this aspect of the invention, a single drape element is attached to the entire circumference of the annular support element. In another preferred embodiment, a plurality of drapes is attached to the support element. In this embodiment, the drapes may be positioned such that there is a small overlap between adjacent drapes, thereby preventing leakage therebetween. This drape sealing feature uniquely solves the problem of PVL following implantation of the replacement valve, since the fabric drape element is capable of moving in response to force applied by the blood flowing during ventricular systole such that it seals most or all of the residual space between the support element and the mitral (or other) valve annulus.

In further preferred embodiments of this aspect of the invention, the annular support element may be fitted with one or more means for reducing PVL, wherein said means are selected from the group consisting of: lateral edge extensions, one or more tubular sealing elements, one or more barbs, an inferiorly-directed circumferential fabric skirt attached to the inner circumference of the ring-shaped support element and an inferiorly-directed fabric curtain attached to the outer circumference of said support element. It is to be noted that the present invention encompasses the use of a combination of more than one of the above mentioned sealing means, or a combination of one of the means with partial use of another means. For example, particularly preferred embodiments include a device with both a tubular sealing element and a fabric skirt, or a tubular sealing element and a partial fabric skirt covering only part of the circumference of the device. Further details of these sealing elements will be provided hereinbelow with reference to the accompanying drawings.

In some embodiments the annular single-ring valve support of the present invention may further comprise one or more lateral extensions as a means for reducing paravalvular leakage as well as for improving the co-axial positioning of the device. These extensions have a surface area which essentially extends the surface area of the ring laterally outwards, to the outer aspect of the ring. The length and width of the extension in the plane of the ring (the lateral plane) are significantly larger than the thickness of the extension, that is, the dimension measured along the longitudinal plane (which is typically only the width of the wire or sheet from which the extension was made). In some embodiments, the extension elements are not continuously connected to the external aspect of the ring along their entire length, but rather are connected to the ring only at discrete singular connection points (for example, connected only at two points, on at the front edge of said element and one at the back edge thereof), without any connection at the central part of the element. In other embodiments, the lateral extensions from a complete crown-like structure around the outer edge of the valve support device. Further details of the lateral extension elements will be provided hereinbelow.

One aspect of the disclosure is a system adapted for endovascular or transapical delivery to replace a mitral valve, comprising: a prosthetic cardiac valve support device comprising a single ring-shaped support element with a collapsed delivery configuration and a deployed configuration, as disclosed hereinabove; and a replacement heart valve comprising an expandable anchor and a plurality of leaflets, wherein said replacement heart valve is adapted to be secured to the prosthetic cardiac valve support device.

In a highly preferred embodiment, the replacement heart valve is a prosthetic aortic valve.

In some embodiments the support element is adapted to securingly engage the replacement heart valve. In one such embodiment, the replacement valve securing means comprise attachment means, such as hooks or other mechanical anchors that are connected, at one of their ends, to the support element, and have a free end for attachment to the replacement valve.

In some embodiments of the invention, the system disclosed hereinabove further comprises pressure measuring elements. These elements may be situated anywhere in the system—including on the surface of the valve support device, attached to the replacement valve, as well as within the guide catheter. In another embodiment, the system of the invention further comprises connection terminals that permit the connection of pacemaker leads to various parts of said system.

One aspect of the disclosure is a method of replacing a patient's mitral valve, comprising: delivering a valve support device to a location near a subject's mitral valve, the valve support device comprising a single ring-shaped support element; expanding the support element from a collapsed configuration to a deployed configuration secured against cardiac tissue above the plane of the mitral valve annulus, below this plane or against the annulus itself;

In one embodiment, the above-defined method may be employed to deliver the valve support device by an endovascular route. In another embodiment, the method may be used to deliver the valve support device by a transapical route.

In some embodiments expanding the support element comprises allowing the support element to self-expand against cardiac tissue.

In certain embodiments, the method further comprises the step of causing cardiac attachment means fitted to the support element to become inserted into the ventricular wall. In certain cases, the insertion of said attachment means is effected by means of control wires inserted through the delivery catheter which are used to cause rotation of the valve support device. In other cases, said attachment means may be covered by a sleeve during insertion of the valve support device, said sleeve being removed in order to allow said attachment means to become inserted into the ventricular wall. In still further embodiments, the attachment means may be constructed in the form of an anchor with two or more backwardly-pointing self-opening distal arms, wherein said distal arms are retained in a closed conformation by means of a resorbable suture. Then, after a certain period of time following insertion of said attachment means into the ventricular tissue (e.g. between a few hours and few weeks), said suture dissolves, thereby permitting the distal arms to adopt their open conformation.

In other embodiments, the above-defined method further comprises the step of causing intra-ventricular stabilizing elements and/or intra-atrial stabilizing elements to engage, respectively, the inner ventricular wall and/or inner atrial wall.

In some embodiments expanding the support element comprises expanding the support element towards a generally annularly shaped deployed configuration.

In some embodiments expanding the support element comprises expanding the support element secured against papillary muscles and chords attached to the native mitral valve, and can be done without displacing them.

Generally, the above-defined method further comprises the subsequent delivery of a replacement heart valve to the location where the ring-shaped valve support device has been implanted, and securing said replacement valve to said valve support device. Securing the replacement heart valve to the valve support can comprise expanding the replacement heart valve from a collapsed delivery configuration to an expanded configuration. Expanding the replacement heart valve can include expanding the replacement heart valve with a balloon and/or allowing the replacement heart valve to self-expand. Securing a replacement heart valve to the valve support can comprise securing the replacement heart valve radially within the valve support. Securing a replacement heart valve to the valve support can comprise locking a replacement heart valve element with a valve support element to lock the replacement heart valve to the valve support. In other embodiments, the step of securing a replacement valve to the valve support device comprises causing valve attachment means fitted to the valve support element to engage said replacement mitral valve. The replacement valve may be delivered by either an endovascular route or by the transapical route.

In one embodiment of this method, the valve support device and the replacement heart valve are delivered by the same route.

In a further embodiment, the above-disclosed method to deliver a valve support device and a prosthetic heart valve may combine two separate delivery approaches—one approach for the support device and a different one for the valve. The advantage of this strategy is that it significantly shortens the time delay between deployment of the valve anchor and the deployment of the prosthetic valve itself. This is important, since after deployment of the valve support there may be interference with the native mitral valve function (due to interference with the valve leaflets). One example of such an approach is the delivery of a valve support via an endovascular, trans-septal route (as described herein), while in parallel delivering the prosthetic mitral valve via a transapical or transfemoral route (as known in the art). Conversely, the valve support may be delivered by a transfemoral or transapical approach, while the replacement valve itself is delivered trans-septally. Thus, in one embodiment of the method disclosed above, the replacement mitral valve is delivered by the same route as the valve support. In another embodiment of the method, the replacement mitral valve and the valve support are delivered by different routes, wherein said routes are selected from the group consisting of trans-septal, transfemoral and transapical. The use of these various approaches to delivery replacement valves and other devices is well known to the skilled artisan and has been described in several publications including U.S. Pat. No. 7,753,923 and WO 2008/070797.

In a preferred embodiment of the method disclosed above, the replacement heart valve used to replace the native mitral valve is a prosthetic aortic valve. Examples of suitable prosthetic aortic valves include (but are not limited to) the following commercially-available replacement valves: Sapien Valve (Edwards Lifesciences Inc., US), Lotus Valve (Boston Scientific Inc., US), CoreValve (Medtronic Inc.) and DFM valve (Direct Flow Medical Inc., US).

As mentioned hereinabove, the method of the present invention is a two-step method for replacing a native cardiac valve, preferably the mitral valve, with a prosthetic valve, wherein the first stage comprises deploying a single-ring valve support device in the region of the native mitral annulus, and the second stage comprises the expansion of an expandable prosthetic valve within the central space of said support device. One of the key advantages of the use of the valve support device of the present invention in this method is that its shape, size and the disposition of its stabilizing arms all permit the native cardiac valve leaflets to continue functioning in the time interval between the deployment of said valve support device (i.e. the first step of the procedure) and the deployment of the replacement valve (i.e. the second step of the procedure). A further key advantage of the presently-disclosed valve support device is that its flat annular form allows the dysfunctional mitral valve to be replaced by a commercially-available prosthetic aortic valve. This is achieved by virtue of the fact that the annular support device is able bridge the gap between the relatively small diameter prosthetic aortic valve and the relatively large diameter native mitral valve annulus. Further advantages of the present invention will become apparent as the description proceeds.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a single-ring valve support of the present invention containing spiral-shaped cardiac anchoring means.

FIG. 2 depicts two support elements, each having the same internal diameter but different external diameters.

FIGS. 3A and 3B illustrate embodiments in which the valve support is fitted with one horizontal stabilizing element (A) and one vertical stabilizing element (B).

FIGS. 4A and 4B depict embodiments of the valve support each having a stabilizing element formed from a stent-like mesh.

FIG. 5 illustrates an embodiment of the valve support in which the stabilizing element contains spring-like constricted regions.

FIG. 6 depicts an embodiment of the valve support having a plurality of stabilizing elements attached to the support element.

FIGS. 7A-7C depict embodiments of the valve support of the present invention in which the stabilizing elements are constructed in the form of curved arms.

FIG. 8 illustrates an embodiment of the valve support in which the support element is connected to a horizontal ring-shaped stabilizing element.

FIGS. 9A and 9B show a valve support device with a pair of elastic tab-like stabilizing elements attached to the support element.

FIG. 10 depicts a valve support comprising a plurality of hook-like cardiac anchors.

FIGS. 11A-11B illustrate cardiac attachment anchors having backwardly pointing distal arms which may be retained in a closed position during delivery by means of a resorbable suture loop.

FIGS. 12A-12B illustrate two different embodiments of cover elements that may be used to conceal the cardiac attachment anchors during delivery of the valve support.

FIGS. 13A-13B depict the use of a shape-memory anchor which is maintained in a straight conformation during delivery by means of an overtube.

FIG. 14 illustrates clip-like cardiac tissue anchors that are particularly suitable for attaching the support element to the annulus.

FIGS. 15A-15B illustrate support elements fitted with a valve engagement means constructed from a soft biocompatible material.

FIG. 16 illustrates an exemplary delivery system for delivering a replacement mitral valve support structure.

FIG. 17 illustrates a support device fitted with two elongate anchoring wings.

FIG. 18 shows the support device of FIG. 17 after the wings have expanded into their open, working position.

FIG. 19 illustrates the valve support device of FIGS. 17 and 18 following its implantation into the heart in the region of the cardiac annulus.

FIG. 20 depicts a different embodiment of the invention, wherein the anchoring arms have enlarged basal sections.

FIG. 21 shows the embodiment of FIG. 22 in its expanded conformation.

FIG. 22 depicts a different embodiment, wherein the anchoring wings are broader than in the previously depicted embodiments.

FIG. 23 depicts a device having two short wings and two long wings.

FIG. 24 shows a support device fitted with an open-work structure.

FIG. 25 depicts an embodiment having an alternative open wing structure.

FIG. 26 illustrates an example of a first implementation of the levered-operated wing embodiment in its expanded conformation.

FIG. 27 shows a similar embodiment to that shown in FIG. 26, but in its pre-expanded conformation.

FIG. 28 provides an enlarged view of a fulcrum point in one embodiment of the lever-operated wings.

FIG. 29 shows a device of the invention fitted with a second implementation of the lever-operated wings in its fully expanded conformation.

FIG. 30 shows the embodiment of FIG. 29 in its pre-expanded conformation.

FIG. 31 provides a perspective view of another embodiment of the second implementation of the lever—operated wings.

FIG. 32 illustrates a third implementation of the lever-operated wings of the present invention.

FIG. 33 depicts a “leaflet pinching” embodiment of the third implementation of the lever-operated wings.

FIG. 34 shows an embodiment comprising both static and levered arms, prior to expansion.

FIG. 35 shows the embodiment of FIG. 34 following expansion of the replacement valve.

FIG. 36 illustrates a top view of an exemplary support element showing the elastic inner perimeter feature.

FIG. 37 illustrates another top view of another exemplary support element fitted with elastically deformable elements.

FIG. 38 illustrates a perspective view of another exemplary support element of the invention fitted with elastically deformable elements.

FIG. 39 illustrates a perspective view of an exemplary replacement valve support fitted with pressure release means, in an expanded configuration.

FIG. 40 provides an illustratory side view of an exemplary support of the present invention, shown in a position on the mitral annulus, and exemplifying a fabric drape attached to the inner part of the ring.

FIG. 41 illustrates a side view of another exemplary upper ring (upper support element) of a valve support of the invention, shown in a position on the mitral annulus, and exemplifying a fabric drape attached to the outer (distal) part of the ring.

FIG. 42 illustrates a perspective view of an exemplary upper ring (upper support element) of a valve support of the invention, exemplifying multiple fabric drapes of the invention.

FIG. 43 illustrates an exemplary design of a fabric of a drape of the invention.

FIG. 44 shows a single-ring support device intended for use in conjunction with a self-expanding aortic valve.

FIG. 45 shows a single-ring support device intended for use in conjunction with a balloon-expandable aortic valve.

FIGS. 46-49 are photographs showing the successful implantation of a single-ring support device of the present invention into a cadaveric heart.

FIG. 50 depicts a wave-like wire spring loop that is suitable for use as a height-increasing element to be attached to a valve support device of the present invention.

FIG. 51 shows a single-ring valve support device of the present invention in which a single wave-like wire spring loop (as depicted in FIG. 50) is attached to the inner circumference of the support ring.

FIG. 52 depicts a hoop-like circular wire spring that is suitable for use as a height-increasing element.

FIG. 53 illustrates a valve support device of the present invention fitted with a wire hoop spring (as shown in FIG. 52).

FIG. 54 shows (in flat form) a single-ring valve support device of the present invention comprising a plurality of tab-like height-increasing elements.

FIG. 55 depicts a valve support device of the present invention, in which the position and direction of undesirable paravalvular flow is indicated by arrows.

FIG. 56 diagrammatical represents a valve support device fitted with lateral edge extensions which function as means for reducing PVL.

FIG. 57 is a photographic representation of a mitral valve support device held within a delivery system, wherein said valve support device comprises a tubular sealing element.

FIG. 58 depicts, in plan view, a valve support device in cut-out form, prior to being crimped within a delivery system, wherein said support device comprises barb-like means that enable improved anchoring of said device within the cardiac tissue, thereby reducing PVL.

FIG. 59 presents a side view of the valve support device shown in FIG. 58, in which the barb-like structures are in their deployed configuration.

FIG. 60 shows a valve support device comprising a full-circumference skirt attached to the inner perimeter of the support ring.

FIG. 61 provides a view of the inferior surface and lateral aspect of a valve support ring having a sealing curtain attached to the outer circumference of the support ring.

FIG. 62 depicts a single-ring valve support device of the present invention fitted with a crown-like lateral extension structure.

### DETAILED DESCRIPTION OF THE INVENTION

The invention is generally related to cardiac valve support structures that are adapted to be implanted near or within a native cardiac valve or native valve annulus and are adapted to provide support for a replacement heart valve. The support structures are adapted to interact with a replacement heart valve to secure it in an implanted position near or within the native valve or native valve annulus. In some embodiments the support structure is adapted to be positioned near or within the mitral valve annulus, and is adapted to interact with a subsequently delivered replacement mitral valve to secure the replacement mitral valve in place to replace the function of the native mitral valve.

The disclosure also provides for two-step endovascular and/or transapical implantation procedures for replacing a patient's native mitral valve. In general, a support structure is first positioned near or within a mitral valve annulus and secured in place. A replacement mitral valve is subsequently secured to the support structure, securing the replacement valve in place near or within the annulus. By implanting the support structure and replacement mitral valve in two steps, the replacement mitral valve can have a lower delivery profile because it does not have to expand as much to contact native tissue due to the presence of the support structure. This eliminates the need to have a large delivery profile replacement valve as would be required if attempting to position an aortic valve in the native mitral valve, or if attempting to position a one-piece mitral valve implant (i.e., an implant not assembled in-vivo) within the native mitral valve.

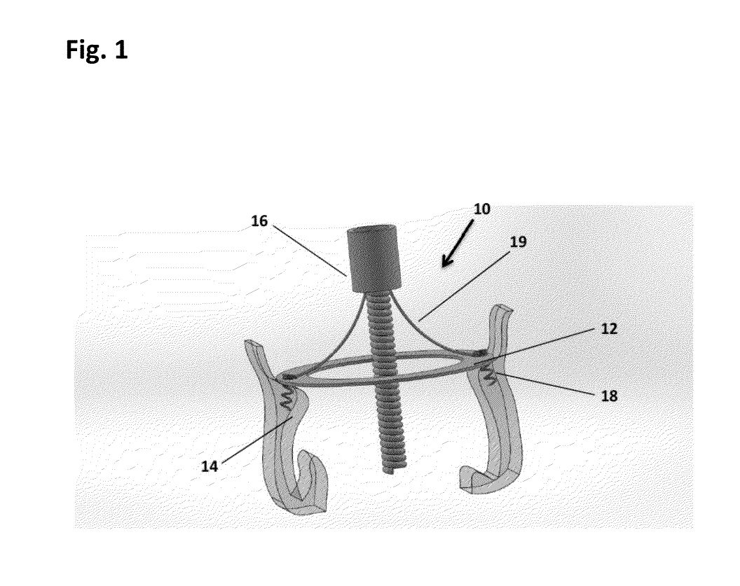

FIG. 1 illustrates an exemplary embodiment of a valve support device of the present invention in an expanded configuration, following its delivery through a guide catheter and implantation at its target site. Thus, FIG. 1 shows a guide catheter 16 that was used to deliver a valve support device 10 of the present invention, wherein said device comprises a single ring-shaped support element 12. At the stage of the delivery process shown in this figure (which will be described in more detail hereinbelow), said support element 12 has self-expanded into its working conformation.

In some embodiments the support element is generally annular in shape in its expanded configurations (see, for example, FIG. 1). Patient-to-patient variability in the cardiac anatomy can, however, require that the support elements have a variety of sizes and configurations. The support elements can therefore have any configuration as needed to be secured to any anatomical configuration. For example, the support elements can have generally elliptical or generally circular configurations.

In some embodiments the support element is made from a resilient material that can be deformed into a delivery configuration yet is adapted to self-expand to an expanded configuration, with optional additional expansion by balloon dilation. For example, the support can be made from Nitinol, relying on its superelastic properties. In some embodiments the valve support is made from a material with shape memory properties, such as nitinol, and is adapted to return to an expanded memory configuration after being heated above its transition temperature. In some embodiments in which the valve support is made from a material such as nitinol, the shape memory properties and the superelastic properties are utilized.

Once the support structure is expanded and secured in place within the native mitral valve, a replacement mitral valve in a collapsed delivery configuration is advanced and positioned within and below the support structure. The replacement mitral valve is then expanded (e.g., by balloon expansion, self-expansion, etc.), thereby causing the replacement mitral valve to engage with, and become secured by, the single-ring support element.

Further details of exemplary deployment procedures are described below.

As disclosed hereinabove, in some embodiments of the support element may be constructed from an annular ring; such a ring may be manufactured from sheets of the shape-memory/superelastic material. In other embodiments, however, the support element is constructed from a shape memory/superelastic wire (such as a Nitinol, cobalt or stainless steel wire). One advantage of this design is the fact that the use of wire results in low manufacturing costs. A further significant advantage is that the use of a single wire (rather than a broader strip—as depicted in FIG. 1) is that it may be collapsed to a very small size such that it may be inserted into a small diameter delivery catheter, thereby presenting a reduced crossing profile.

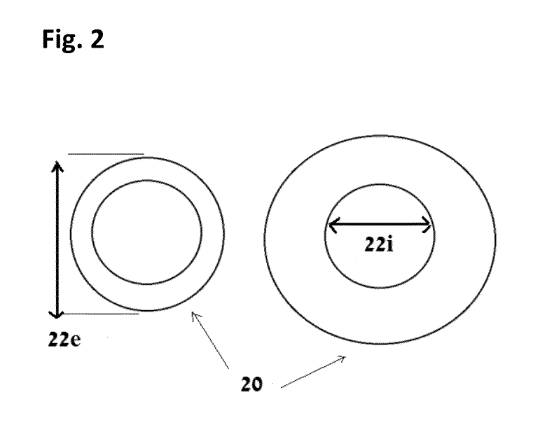

In the embodiment of the valve support disclosed herein in which said support is constructed in the form of an annular ring (such as shown in FIG. 1), the size of the ring-like support element may, as depicted in FIG. 2, be defined by two different dimensions—an external diameter 22e and an internal diameter 22i. It will be seen that while both of the support elements 20 shown in this figure have the same internal diameter, their external diameters differ.

It will be appreciated that the internal diameter defines the space available for implantation of the replacement valve within the valve support device, while the external diameter needs to be the same as the space within the native Mitral annulus (in order to permit stable implantation of the valve support). Since both the expanded diameter of different commercially-available replacement mitral valves and the diameter of the anatomical mitral annulus differs (from patient to patient), it follows that a range of valve support devices needs to be manufactured and made available, such that the clinician can select the valve support having an internal diameter appropriate for the replacement valve to be implanted and an external diameter of the same size as the space within the mitral annulus.

In the embodiments described herein the support elements do not have a covering element. In some embodiments, however, the support element can have a covering element such as a sealing skirt to enhance the sealing of blood flow in and around the support structure and replacement heart valve. The covering element can be any type of material that surrounds the support element and provides the enhanced sealing functionality (e.g. it can prevent fluid leakage between the valve support and the heart wall). In some embodiments, the covering element can be attached (e.g. by the use of a biocompatible adhesive) to the outer surface of the support element. In other embodiments, the covering element can be attached to the inner surface of the support element.

In some embodiments one or more of support structures is covered in a material such as a polyester fabric (e.g., Dacron).

In certain embodiments, the valve support device may further comprise one or more stabilizing elements attached to the support element. The purpose of the stabilizing elements is to increase the multi-directional stability of the implanted valve support device (and thus also enhance the stability of the implanted replacement valve), by means of stabilizing elements in the form of additional complete ring structures, partial rings or curved arms, elongate arms or wings and levered arms or wings, whereby said structures are placed such that at least part of their length is in close apposition to the surface of the inner ventricular wall and/or the surface of the inner atrial wall. Since the curvature of the inner walls of both the atrium and ventricle may be defined in relation to two mutually-perpendicular axes (horizontal and vertical), the stabilizing elements may be disposed either horizontally (i.e., essentially parallel to the horizontal axis of the valve support device) or vertically (i.e. essentially parallel to the vertical axis of the valve support device.). Additionally, in some embodiments, the stabilizing elements may be disposed such that they are neither parallel to the horizontal axis nor to the vertical axis, but rather are arranged at an acute angle to one of these axes.

In some cases, the stabilizing elements (which may be formed from either elastic or plastic materials, as will be described hereinbelow) will be manufactured as an integral part of the valve support device. In other cases, said stabilizing elements will be manufactured separately (by casting, milling, laser-cutting or any other suitable technique known to skilled artisans in the field), and later connected to one or both support elements by means of soldering or laser welding.

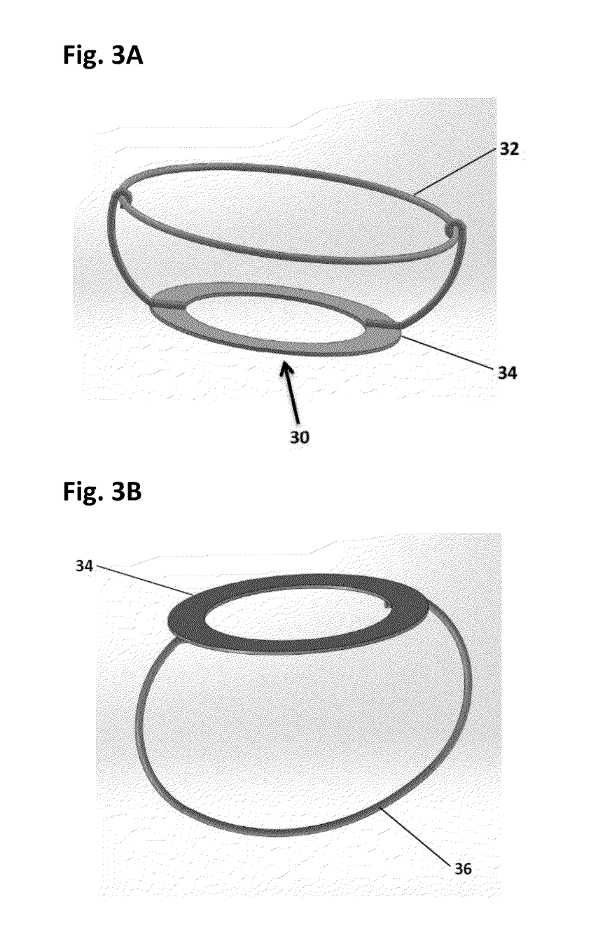

FIG. 3A illustrates a valve support device 30 of the present invention fitted with a single, horizontally-disposed ring-shaped stabilizing element. As shown in the figure, the upper, apical stabilizing element 32 is attached at its lower portion to the support element 34. FIG. 3B depicts another embodiment of the valve support device of the present invention in which a single, vertically aligned ventricular stabilizing element 36 is attached at its lower portion to support element 34. In use, the upper stabilizing element shown in FIG. 3A will be in contact with the inner atrial wall, while the lower stabilizing element of FIG. 3B will contact the inner ventricular wall.

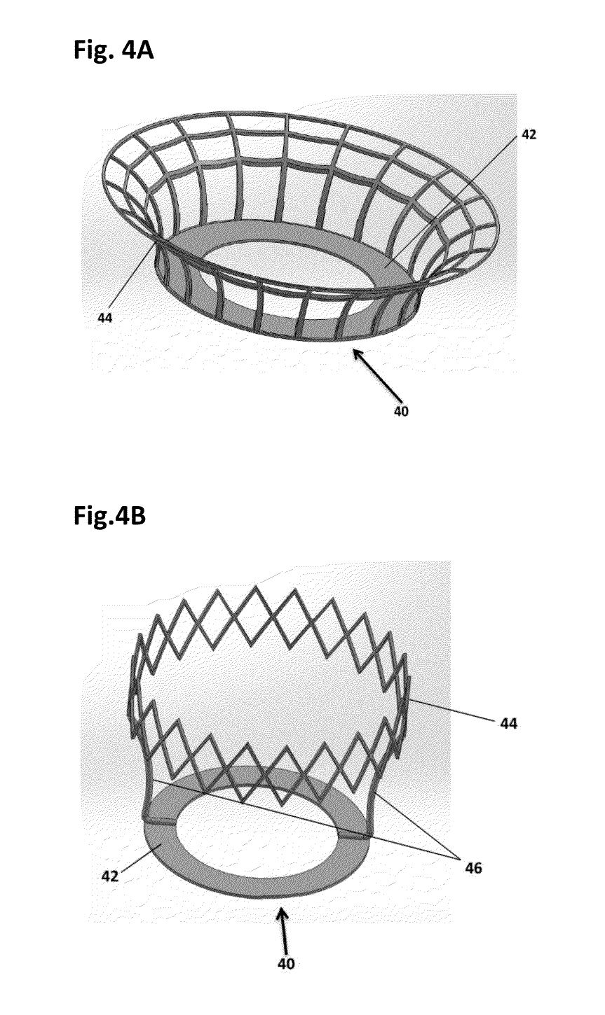

In the case of horizontal stabilizing elements, the element itself can (as explained above) be a complete ring, a partial ring or a curved elongate arm. While in some complete ring embodiments, the stabilizing element is constructed from a single looped wire or solid band, in other embodiments, it may be constructed in the form of a stent-like mesh. FIG. 4A illustrates one embodiment of this type, in which the mesh-like stabilizing element 44 is attached directly to the support element 42 of valve support device 40. Alternatively, as shown in FIG. 4B, the mesh-like stabilizing element 44 may be connected to the support element 42 by means of spacer arms 46, which serve to increase the separation distance between the stent-like mesh stabilizer 44 and said support element 42.

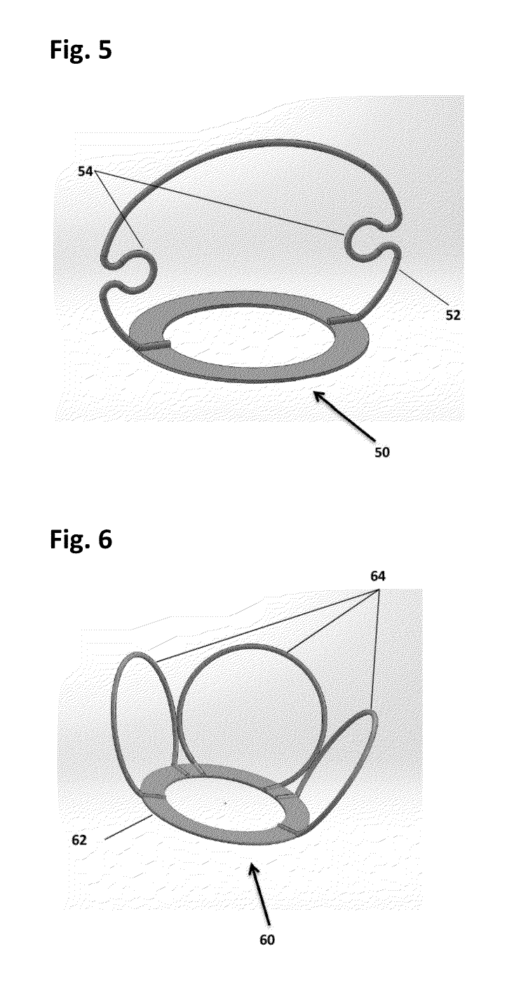

While the stabilizing element is generally constructed such that its outline shape is that of a smooth curve, in another preferred embodiment, as depicted in FIG. 5, this smooth curve is broken by one or more constricted regions 54, wherein said regions act as spring-like elements, increasing the force that the stabilizing element 52 is capable of applying to the inner ventricular or atrial wall, and thereby enhancing the ability of said stabilizing element to stabilize the valve support device 50. The device shown in FIG. 5 contains a single, upper (apical) vertical stabilizing element. However, in other versions of this embodiment, the valve support device may be fitted with one or more vertical stabilizing elements and one horizontal stabilizing elements attached to the other support element. In some other embodiments, the valve support device contains only one such stabilizing element (horizontal, vertical or otherwise angled). In still further embodiments, a single valve support device may contain one stabilizing element containing one or more constricted regions 54, as shown in FIG. 5, together with one or more stabilizing elements of any of the other types disclosed and described herein.

All possible combinations of the various types of stabilizing element disclosed herein may be used, as appropriate. It should also be noted that more than one stabilizing element may be attached to the support element. FIG. 6 illustrates one embodiment of this type, in which the support element 62 of the valve support device 60 is fitted with several (in this case, three) non-horizontal, angled, atrial stabilizing elements 64.

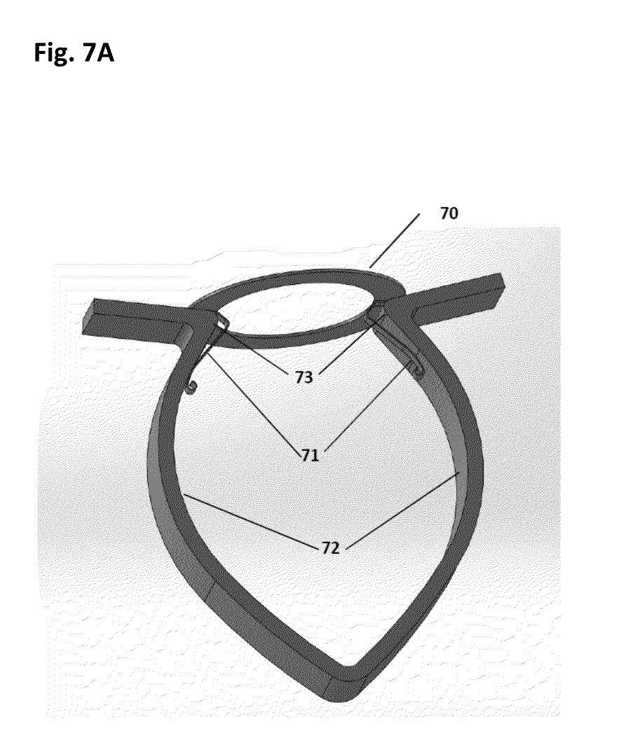

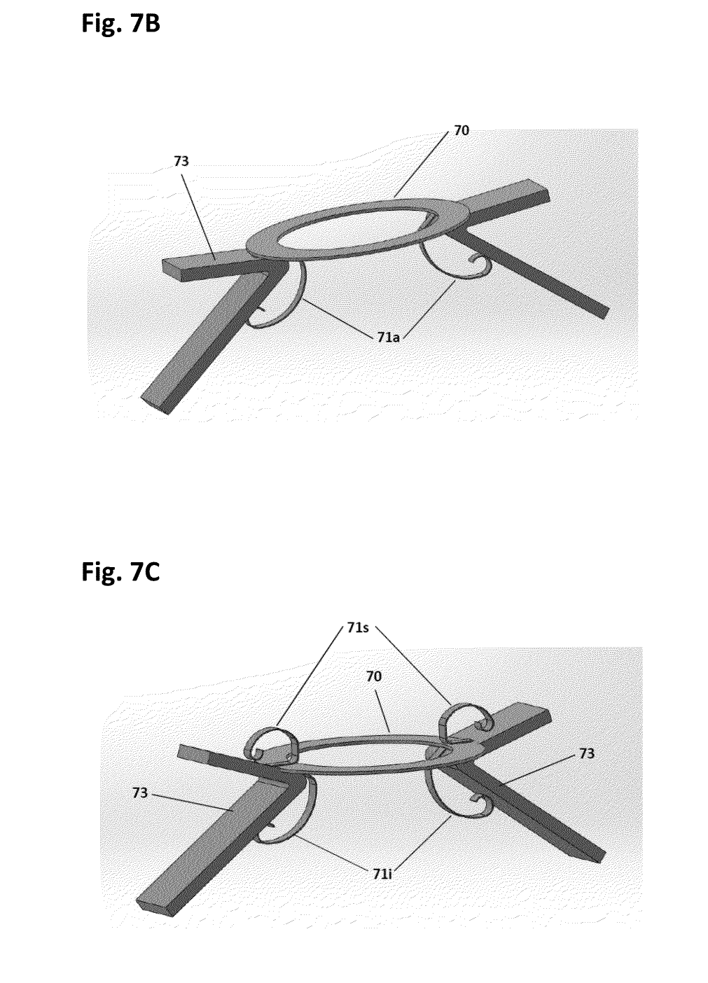

As explained hereinabove, the stabilizing element need not be provided in the form of a complete ring, but rather may also have the form of a partial ring or a curved elongate arm. Various examples of the latter type of stabilizing element are shown in FIGS. 7A, B and C. Thus, FIG. 7A depicts a support element 70 of a valve support device of the present invention, wherein said valve support device is connected to—and stabilized by—two curved elongate arms 71 which are disposed vertically downwards along the inner ventricular wall 72. In the example shown in this figure, the stabilizing elements 71 are constructed from an elastic material (such as cobalt base alloy, nitinol, stainless steel and other biocompatible metals and metal alloys). The curved arms typically have a length of between 1 mm and 50 mm, preferably about 20 mm. As will be seen in the figure, the upper part of each stabilizing element 71 is angled such that it is able to pass around the cardiac annulus 73. In some embodiments, the elongate, curved elastic arms may be constructed such that they are in a state of pre-load. The elastic properties of the stabilizing elements will cause said element to tend to both grip the annulus and to apply an outward force on the ventricular wall inferior to the annulus. In an alternative embodiment of this aspect of the invention, the curved elongate stabilizing elements may be constructed from a plastically-deformable material such as stainless steel, cobalt base alloy and nitinol. In this case, the elongate arms are molded around the annulus using a clenching or crimping tool. In this way, the upper sections of the elongate arms will firmly grip the annulus, while the lower sections will be biased outward and downwards along the ventricular wall.

FIG. 7B illustrates another embodiment of this aspect of the device, wherein the stabilizing elements 71a attached to support element 70 are much shorter than those shown in FIG. 7A, and apply a stabilizing force to the inferior surface of the annulus 73 (rather than to the lateral inner walls of the ventricle). During implantation, the stabilizing elements are brought into position below the annulus, such that the annulus becomes “trapped” between said stabilizing elements and the support element itself.

A still further variant of this embodiment is illustrated in FIG. 7C. This variant differs from the embodiment shown in FIG. 7B, in that the support element 70 is fitted with both upper (71s) and lower (71i) stabilizing elements. During implantation into a patient, the valve support device is manipulated such that the annulus 73 becomes “trapped” between these upper and lower stabilizing elements. In each of the variants of this embodiment, the short stabilizing elements may be brought into position by means of a balloon expansion mechanism, by a mechanical closure mechanism or, alternatively, said stabilizing elements may be self-expanding.

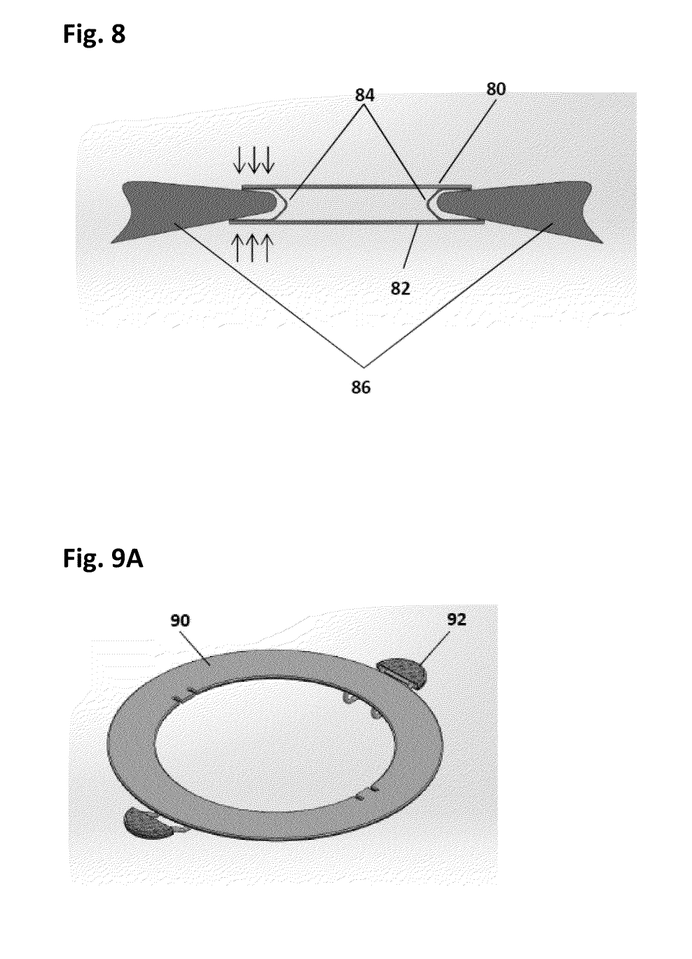

FIG. 8 depicts an alternative design of the valve support of the present invention, additionally comprising a horizontally-disposed ring-shaped stabilizing element 82, located inferior to the support element 80. Elastic members 84 mutually connect support element 80 and said horizontal stabilizing element 82. The annulus 86 may thus become trapped or pinched between them (as indicated by the arrows). This design may either be used without any additional stabilization elements, or in combination with any of the stabilization element embodiments described hereinabove.

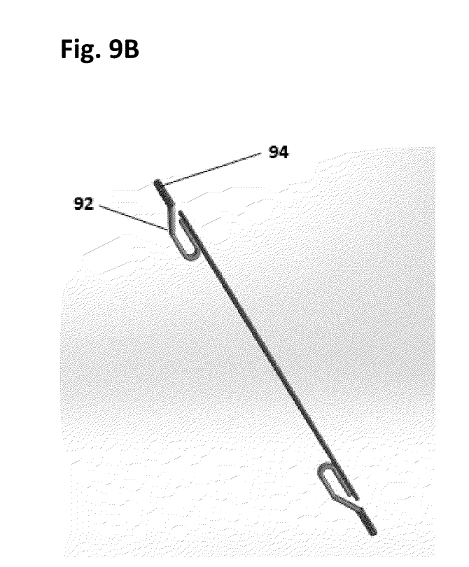

In a still further embodiment, as depicted in FIG. 9A, the valve support device as viewed from above is seen to comprise a pair of elastic stabilizing elements 92, one on each side of the support element 90. These stabilizing elements may be manufactured from biocompatible metals including (but not limited to) Nitinol, Cobalt and Stainless steel, and are manufactured in the form of a spring-like tab that permits the elastic forces applied by the device on the ventricular wall to be distributed over a large surface area, so as to minimize local pressure on the cardiac tissue, thus minimizing the danger of necrosis of cardiac tissue due to high-level mechanical stress. The structure of the tab-like stabilizing elements 92 may be better seen in the side view of this embodiment of the device, presented in FIG. 9B. As may be seen from these figures, each tab may preferably be covered by a biocompatible fabric or mesh 94 (for example made from Dacron, PTFE etc.), the key functions of which are to assist in distributing the force, as previously explained, and also to encourage growth of cardiac tissue on the device, thus improving the attachment thereof to the heart wall. One particular advantage of using this type of stabilizing element is that it approximates the support element to the floor of the left atrium, thus essentially compressing the annulus (the stabilizing element compressing from the ventricular side and the support element compressing from the atrial side), thereby forming a “plug” that will prevent paravalvular leakage, even in cases in which the annulus is larger in diameter than the prosthetic valve, provided that the support element is larger than the annulus. In this embodiment, the support element may be fitted with one or more stabilizing elements of this type, which may be distributed evenly or unevenly around the circumference of said support element. Exemplary dimensions of this tab-like stabilizing element are as follows: width 2-20 mm; and length 2-20 mm. However, it is to be recognized that these measurements are for the purposes of illustration only, stabilizing elements of dimensions larger or smaller than these ranges being included within the scope of the present invention.

As explained hereinabove, the stabilizing elements of the present invention may be provided in the form of elongate anchoring wings, cut out of the same metallic disk used to manufacture the single-ring support device. An example of a single ring support structure comprising two anchoring wings of this type is illustrated in FIG. 17. (It will be appreciated that this figure—as well as all similar figures exemplifying top views of similar devices—are intended to show said devices in their pre-crimped conformation.) The support structure 710 in this example is seen to comprise a circular support ring 712 fitted with elements 714 which permit the inner circumference of said ring to elastically deform in a radial direction (thereby facilitating the precise adaptation of the ring to a replacement valve of any size). The device also comprises two anchoring wings 716, the basal sections 718 of which are continuous with the ring itself. Indeed, in most preferred embodiments, the wings have been cut out of the same disk as the ring itself. Finally, each of said wings also has a small aperture 719 formed close to its distal tip, the purpose of said aperture being assist the operator in gripping the support device during implementation, as will described in more detail, hereinbelow.

FIG. 18 shows the same valve support device following its release from the delivery catheter, and after the anchoring wings 820 have expanded into their open, working conformation.

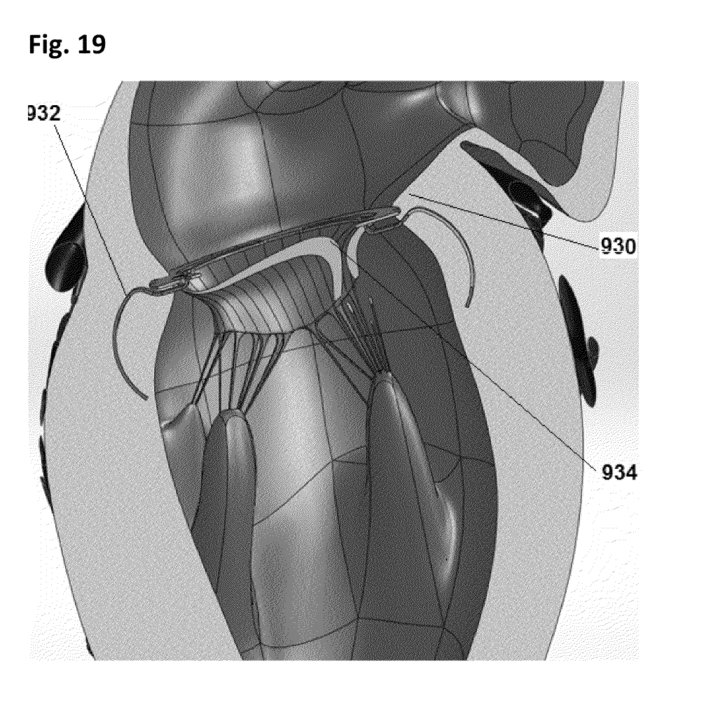

FIG. 19 illustrates the valve support device of FIGS. 17 and 18 following its implantation into the heart in the region of the cardiac annulus 930. Thus, it will be seen that the anchoring wings 932 are aligned along the commissure of native mitral valve 934, such that the presence of the support device does not interfere with the functioning of said native valve at this stage. It is to be noted that the anchoring wings 932 compress the ventricular tissue with which they are in contact, thereby causing a slight radially-outward displacement of said tissue. (This displacement is not visible in FIG. 19, due to drawing limitations.)

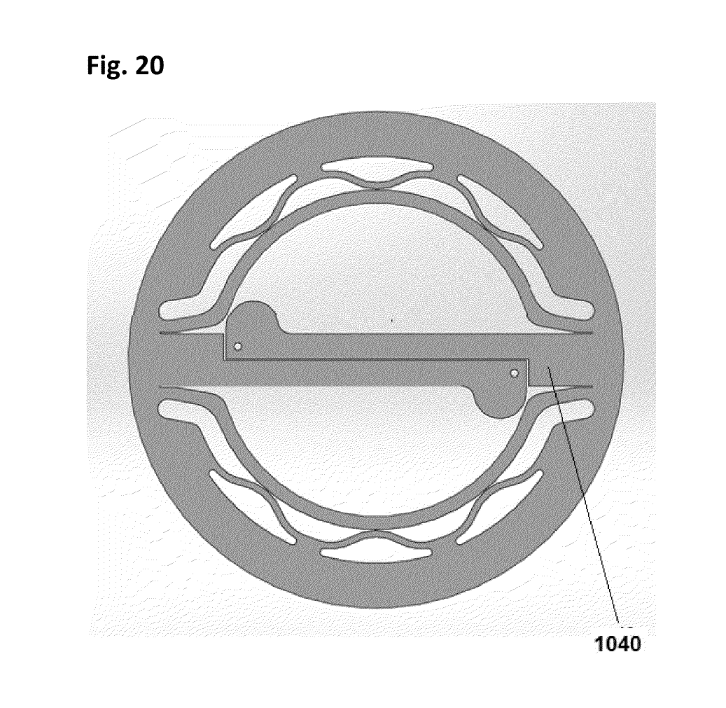

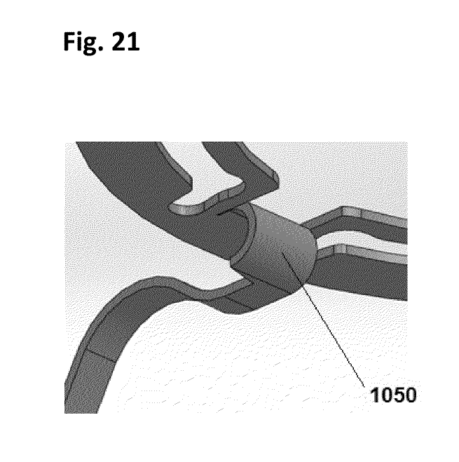

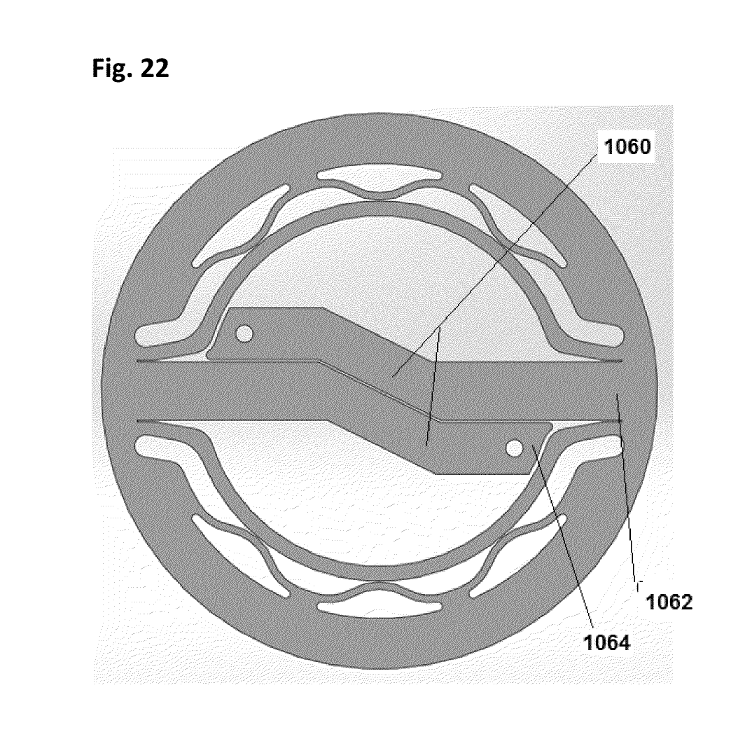

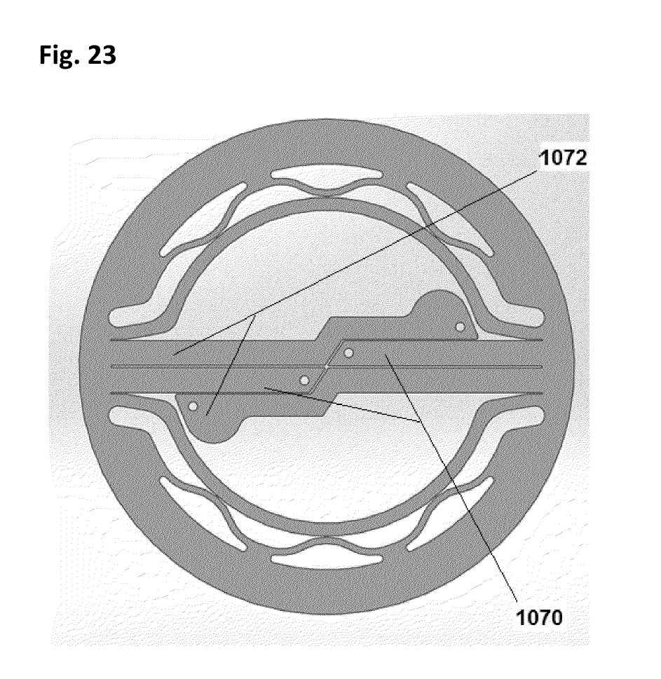

A different embodiment of this aspect of the invention is illustrated in FIG. 20, in which it may be seen that each anchoring wing has an enlarged basal section 1040. It may be further seen in the enlarged side view of this device in its expanded conformation (shown in FIG. 21), that the expanded basal section (now shown as 1050) contributes to the mechanical strength of the anchoring wing precisely at the point where said wing curves away from the ring support structure.

In yet another embodiment, as shown in FIG. 22, the anchoring wings 1060 are broader than the wings depicted in the earlier drawings, this increased breadth being maintained along the entire length of each of said wings, from the basal section 1062 to the distal tip 1064. As a consequence of their greater breadth, the anchoring wings of the embodiment depicted in this figure are able to transmit a greater stabilizing force onto the ventricular tissue. This larger wing also distributes the anchoring force on a larger surface area of the heart—this is beneficial since force distribution reduces the local stress on myocardial tissue, and this may be clinically beneficial since it will prevent high stresses that may damage tissue.

A slightly different approach is shown in FIG. 23, in which the support device comprises four anchoring wings—two short wings 1070 and two long wings 1072 which are disposed such that one short wing and one long wing are situated side-by-side on each side of the device. One advantage of this embodiment of the support device is that the presence of both a short wing and a longer wing on each side forms a compensatory mechanism such that in the event that one of said wings (e.g. the long wing) on each side does not make satisfactory contact with the ventricular wall, then the other one (the short wing) will be able to do so.

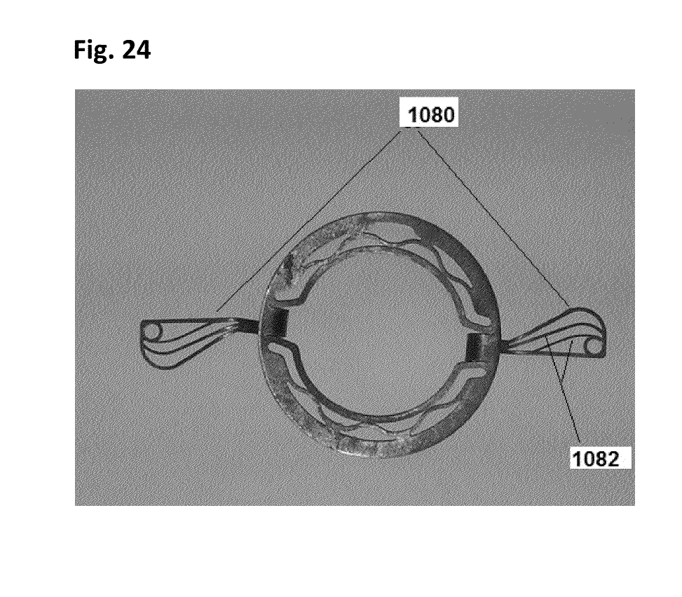

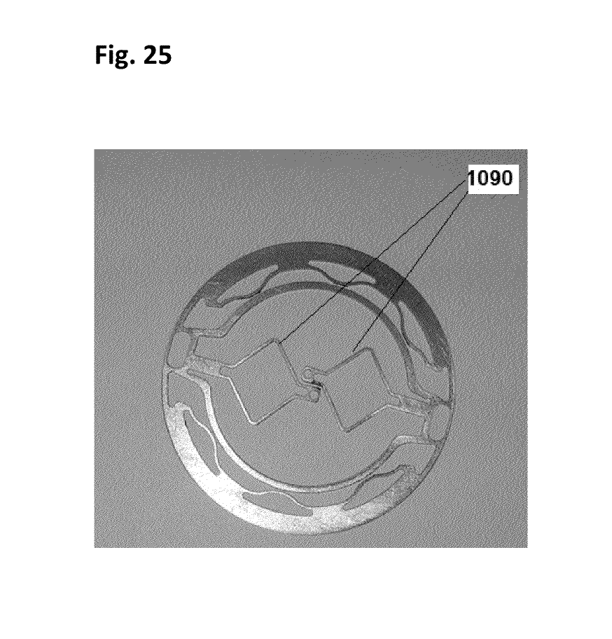

In all of the various embodiments described thus far and depicted in FIGS. 17 to 23, the anchoring wings are formed as solid structures cut out of the same disk as the support ring itself. In an alternative approach, as shown in the photographic view presented in FIG. 24, the wings 1080 are constructed as open structures. This type of wing may be created, for example, by means of first cutting out a broad wing from the support ring disk, and then further removing material, such that one or more metallic strands remain within the wing. Two such strands 1082 are shown in the design depicted in FIG. 24. One advantage of this approach is that broader anchoring wings may be constructed (thereby being able to apply stabilizing forces to a larger area of the ventricular wall), without adding to the bulk or weight of said wings. As previously explained, this larger wing also distributes the anchoring force on a larger surface area of the heart—this is beneficial since force distribution reduces the local stress on myocardial tissue, and this may be clinically beneficial since it will prevent high stresses that may damage tissue.

A further embodiment is shown in the photograph presented in FIG. 25. The device shown in this figure comprises wings having an open structure that are capable of existing in two different conformations—(a) an elongated, small-diameter conformation that is created during crimping during the insertion of the device into the delivery catheter and (b) a shortened, broad form, as shown in FIG. 25. As shown in the figure, the anchoring wings 1090 of this specific embodiment, in their working conformation, have a broad, diamond-like shape, and are thus capable of exerting relatively high stabilizing forces on regions of the ventricular wall close to the support device. It is to be noted that if wings having this enlarged breadth were to be formed as solid structures, it would be very difficult to crimp the device into its collapsed, delivery, conformation. Thus, the use of a skeleton structure of the type shown in this figure is highly advantageous since it combines the advantages of long, narrow wings for catheter delivery with the mechanical advantages of short, broad wings once the support device has been deployed.

The wings may be covered—either completely or, alternatively, at their distal tips only—with a fabric or other covering material. In one highly preferred embodiment, a covering material, such as biocompatible Dacron, that will permit ingrowth of cardiac tissue thereinto, is used. In this way, additional anchoring of the wings to the cardiac tissue may be achieved.

The devices may be produced by laser cutting of the Nitinol disks that are used to create the support devices. The rings are then subjected to heat treatment (at temperatures of, for example, 500-600 degrees C.) with the wings bent in the desired working position, such that following release from the delivery device, the wings will adopt this new shape-memory position.

In some preferred embodiments, the wings will have small holes drilled through their distal-most portions, in order to allow the operator to easily grip the support device with a narrow-ended tool or wire during release from the delivery catheter, thereby facilitating the maneuvering of said device into its working position.

As disclosed hereinabove, in another group of preferred embodiments, the stabilizing elements may be provided in the form of lever-operated wings or arms. In a first implementation of the present invention, the valve support device comprises an upper, single-ring valve support device connected by means of two or more bridging elements to a lower fulcrum support ring. The valve support device in this implementation further comprises two or more anchoring arms (i.e. the same number of anchoring arms as the number of bridging elements), each of which is bent at a point along its length (as explained hereinabove) thereby defining a medial anchoring arm portion and a lateral anchoring arm portion. One end of each anchoring arm is attached to the upper (i.e. valve support) ring close to the point at which one of the bridging elements is attached. The opposite extremity of each anchoring arm is unconnected to any other structure in the device. The anchoring arms are disposed such that either the medial portion or the lateral portion thereof passes laterally through an aperture in the adjacent bridging element. Although said aperture may be formed in any convenient shape, in a preferred embodiment of this aspect of the invention, the aperture is rectangular. Either the inferior side or the superior side of said aperture acts as a fulcrum about which the anchoring arm is able to pivot.

In one preferred embodiment of this implementation of the device, the fulcrum support ring is provided in the form of a thin wire (for example, a Nitinol wire having a diameter of 0.4 mm). In this embodiment, the wire “ring” is in a contracted state, and takes the form of a stirrup (rather than an open ring) prior to lateral expansion of the anchoring arms. One advantage of this contracted form is that it does not interfere with native valve leaflet function during the first step of the two-step implantation procedure. Also, the minimal surface area presented by this contracted form facilitates expansion of the stented-valve in the second step of said procedure. As the expansion of the stented-valve proceeds, the forces applied thereby onto the contracted, stirrup-shaped fulcrum support element causes said element to adopt its open ring conformation.

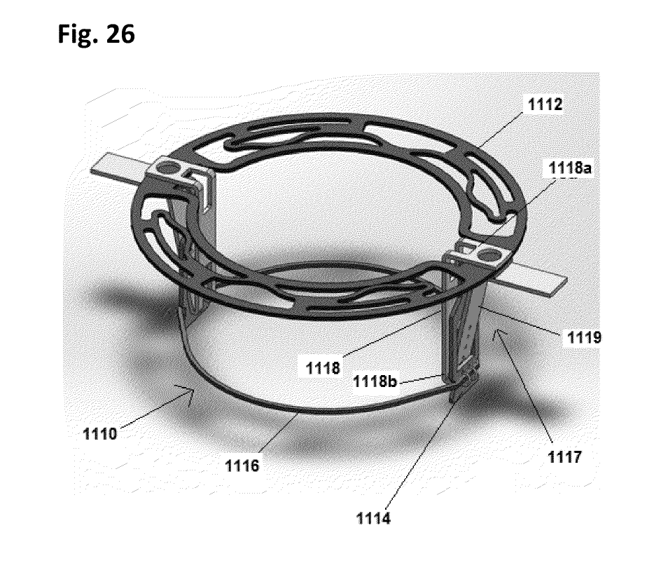

An example of this embodiment of the invention shown in its fully-expanded conformation is depicted in perspective view in FIG. 26, generally indicated as 1110, which comprises a valve support ring 1112, connected by two bridging elements 1114 to a lower fulcrum support ring 1116 which is constructed in the form of a thin Nitinol wire. The device comprises two anchoring arms 1117, the medial portion 1118 of each one having an upper end 1118a that is attached (e.g. welded) to the upper support ring, and a lower end 1118b that ends in sharply-angled portion. The lateral portion 1119 of each anchoring arm then passes upwards and outwards from the angled portion, passing through a rectangular opening in bridging element 1114. In the embodiment shown in this figure, the terminal portion of the distal end of lateral anchoring arm portion 1119 is angled at approximately 90 degrees to the rest of said lateral portion. However, this terminal portion may also be constructed in a variety of different forms.

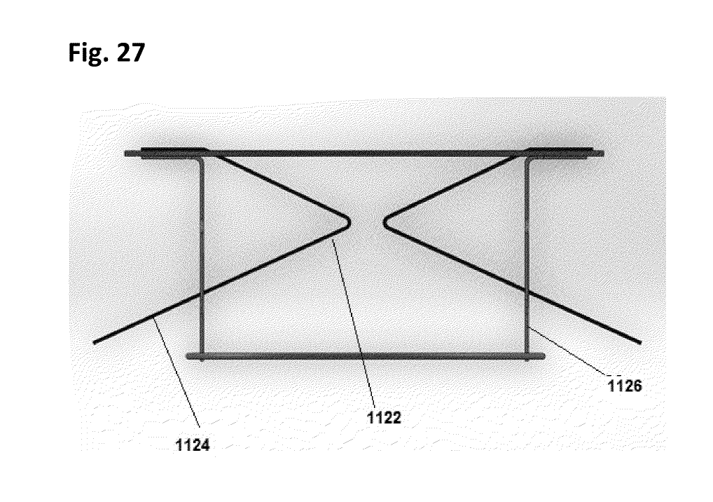

FIG. 27 provides a side-view of a device very similar to that presented in FIG. 26, but in its pre-expanded conformation. It will be seen from this figure that the angled portions 1122 of each of the two anchoring arms, are initially located close to each other, within the central space of the valve support device. Then, after implantation and expansion of the replacement valve (during the second stage of the replacement procedure), the expanding valve applies pressure to the angled portions, causing them to move laterally, while each the lateral portions 1124 of the anchoring arms pivots around its fulcrum point, which is provided by the lower edge of the rectangular opening in bridging element 1126.

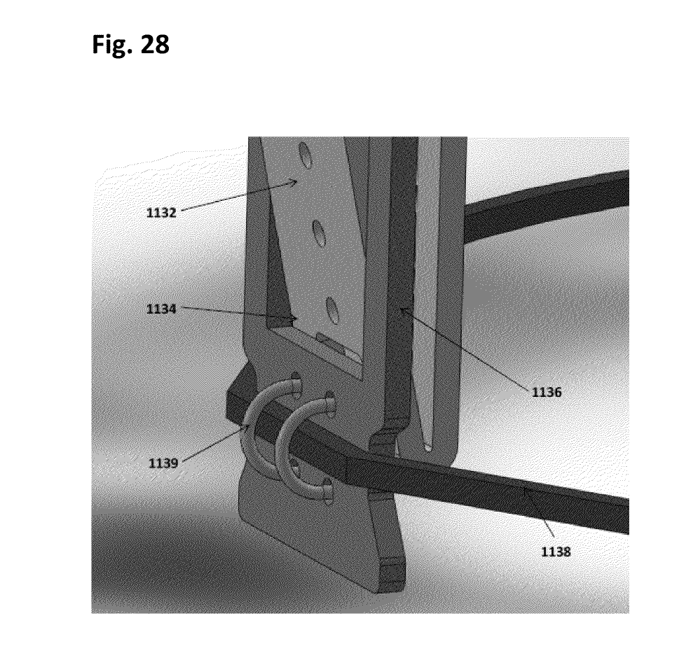

As mentioned above, in this embodiment, the lower edge of said rectangular opening acts as the fulcrum for the levered anchored arm. An enlarged view of the fulcrum point is shown in FIG. 28, in which it may be seen that the lateral portion 1132 of the anchoring arm on one side of the device is in contact with—and capable of pivoting around—the lower margin 1134 of the rectangular opening in bridging element 1136. This figure also illustrates one way in which the bridging element 1136 may be connected to the fulcrum support ring 1138, namely by means of small wire staples or loops 1139.

In a second implementation of the present invention, the valve support device comprises a single-ring support element and (similar to the first implementation) further comprises two or more anchoring arms, the superior ends of which are attached to said upper support ring. In addition, the valve support device further comprises a lower ring element that is similar to the stirrup-shaped element described in connection with one of the preferred embodiments of the first implementation, hereinabove. However, in contradistinction to the first implementation, the presently-described implementation does not comprise bridging elements connecting said stirrup-shaped element to the upper support ring. Rather, each stirrup-shaped element is connected directly to each of the anchoring arms.

Functionally, this implementation differs significantly from the first implementation described above, since when the support device is in its rest position (i.e. before radial expansion) there is no fulcrum about which the levered anchoring arms are able to rotate. Rather, the fulcrum is created only after the stirrup-shaped wire is expanded (by means of the pressure applied by the expanding stented replacement valve). At a certain point, the lower wire element becomes ring-shaped. At this point, the lower wire element is unable to expand any further, and the point of attachment of each anchoring arm to the lower wire element now functions as a fulcrum, about which said anchoring arms rotate in response to the radially-outward force generated by the expanding replacement valve. It may thus be appreciated that while in the first implementation (described above), the fulcrum is present at all stages (from pre-expansion to full expansion), there is no fulcrum in the second implementation until the lower wire element has been fully expanded into its ring conformation.

In one preferred embodiment of this implementation, the device comprises two anchoring arms which are attached to the support ring (and to the lower wire element) at points separated by approximately 180 degrees from each other (as measured along the circumference of the upper support ring). In this embodiment, the valve support device is intended for implantation into the mitral valve annulus such that the anchoring arms are disposed along the valve commissure such that they do not interfere with native valve leaflet function during the first stage of the two-stage implantation procedure. In addition, the lateral portions of said anchoring arms are shaped such that they may be used to apply axially-directed forces on the ventricular wall (as described above, in relation to the first implementation of the device).

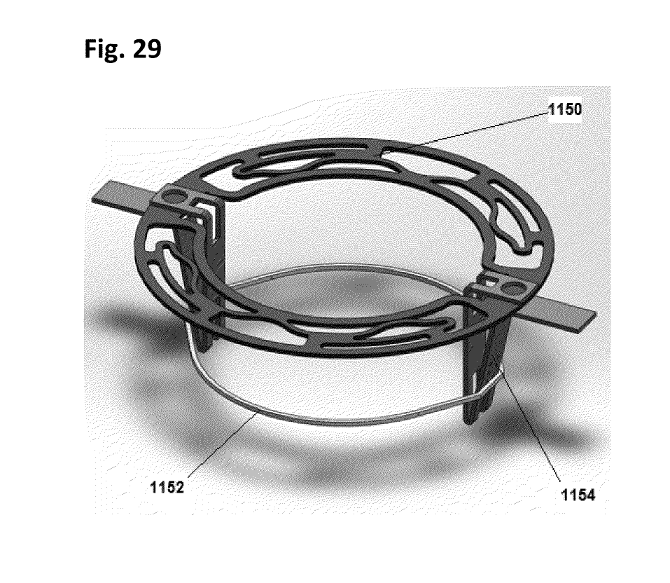

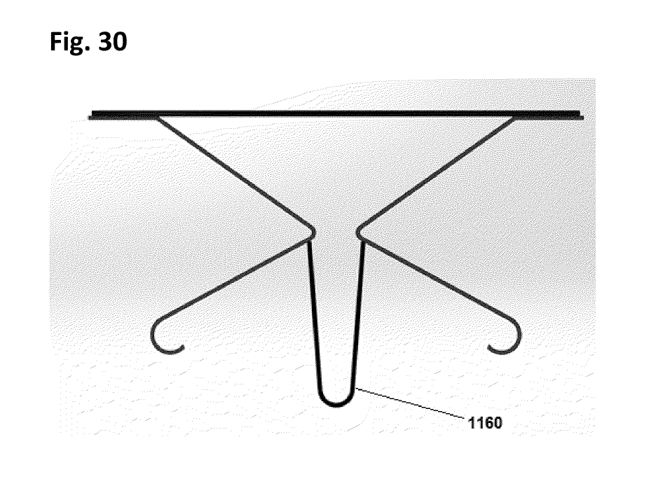

An example of a device of this type is shown in FIG. 29, which provides a perspective view of the device in its fully expanded position. As explained above, the device comprises a valve support ring 1150 and a lower fulcrum support ring 1152, which, in its pre-expanded conformation has a stirrup-like shape (see FIG. 30). The anchoring arms 1154 are immovably attached to said support ring (e.g. by means of welding) and pivotably attached to lower ring/stirrup 1152 by, for example, small rings or staples (not shown for clarity).

A device of this implementation, similar to that illustrated in FIG. 29, is shown in its pre-expanded conformation in side view in FIG. 30. It may be seen from this drawing that the lower fulcrum support ring 1160 is, in this conformation, stirrup-shaped and is very compact, thereby offering no resistance or interference to native valve function.

In another preferred embodiment of this implementation, the device comprises two or more anchoring arms which are constructed such that when they are in their laterally-expanded position, the angle between the medial and lateral portions of said arms is very small, such that said portions are almost in mutual contact. The small space between these portions may then be exploited in order to ‘pinch’ the native valve leaflets, thereby maintaining them in a fully-displaced, fully-open disposition. It may be appreciated that in this embodiment, anchoring and stabilizing of the support device is achieved by virtue of the fact that the anchoring arms firmly grip the valve leaflets which are in turn anchored to the ventricular wall tissues by means of the chordae tendineae and underlying papillary muscles. In one particular version of this embodiment, the leaflet-pinching effect exerted by the anchoring arms may be enhanced by the use of multiple prongs fitted to the inner surface of one of the portions (medial or lateral) of the anchoring arm, which are capable of penetrating the tissue of the entrapped valve leaflets upon lateral expansion of said arm, and becoming locked into correspondingly located and sized apertures on the inner surface of the other portion thereof.

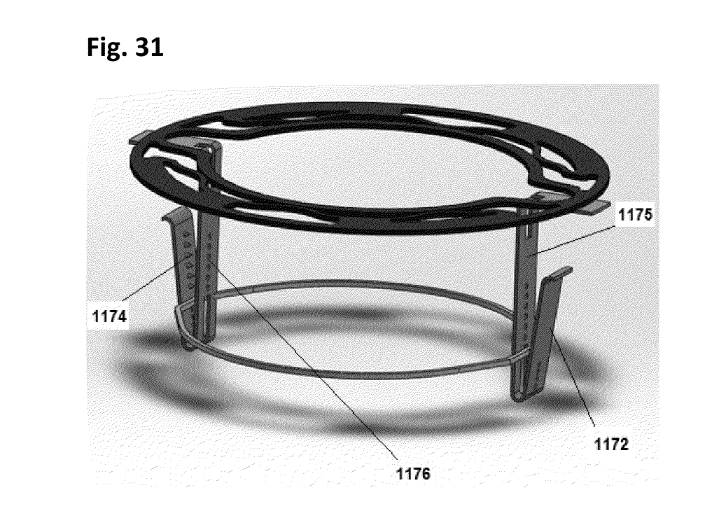

An example of this embodiment of the second implementation of the invention is shown, in perspective view, in FIG. 31. As explained hereinabove, the inner surface of one of the portions of each of the anchoring arms—in this case the lateral portion 1172 is fitted with a plurality of sharp prongs 1174. The medial portion 1175 of each anchoring arm in this particular embodiment comprises a set of small apertures 1176 which correspond in position and size with said prongs 1174. In use, following expansion of the replacement valve (in the second step of the two-step replacement procedure), the lateral portion 1172 of each of the anchoring arms is manipulated such that one of the native valve leaflets is trapped or ‘pinched’ between it and the medial portion 1175 of the same anchoring arm, and firmly held in place by prongs 1174 which penetrate the leaflet tissue and become anchored within apertures 1176.

This implementation of the device of the invention thus possesses, inter alia, the following advantages:

The absence of bridging elements leads to a valve support structure that contains less material, and is therefore cheaper to construct, causes less interference with the native valve function and results in easier crimping of the device during its insertion into the delivery catheter.

The absence of bridging elements is further advantageous since there is now no need to align the anchoring arms (which in the first implementation were attached to said bridging elements) such that they are located along the valve commissure. Rather, the anchoring arms may (in one embodiment) be aligned such that each of the native valve leaflets becomes ‘pinched’ by the medial and lateral portions of one of the anchoring arms.

The fulcrum is created precisely when the lever effect is most needed—that is, at the point when the expanding replacement valve has caused maximum lateral displacement of the native mitral valve leaflets.

In the first two implementations of the device disclosed and described hereinabove, the support device becomes anchored to the ventricular wall only during and after expansion of the stented replacement valve. In a third implementation of the present invention, however, the valve support device comprises anchoring arms which are capable of applying both weak forces to the ventricular wall during the first stage of the two-step implantation procedure and then stronger forces during the second stage of said procedure. In order to achieve this technical effect, the device, in this implementation comprises a single-ring valve support element to which are attached two or more curved anchoring arms that are essentially devoid of straight portions.

In one preferred embodiment of this implementation, said curved anchoring arms initially curve in an inferio-medial direction (i.e. towards the center of the internal space of the support ring. Then, the direction of the curvature of said arms changes such that they curve inferio-laterally, laterally, superio-laterally and then in a superior direction, finally ending in a short portion that curves back in a medial and inferio-medial direction. In this particular embodiment, the curved anchoring arm has an outline form similar to an uppercase ‘D’ letter, with the flatten portion of the ‘D’ being represented by the upper portions of said arm. During the first stage of the implantation procedure, the curved arms are capable of applying relatively weak stabilizing forces to both the lateral wall of the ventricular cavity, as well as the tissue forming the roof of said cavity. Then, during and following expansion of the stented replacement valve within the central cavity of the support ring, the curved arms of said ring are pushed outwards and (as result of their curvature) upwards, such that said arms are capable of exerting much stronger forces on the lateral and superior walls of the left ventricle. In addition, the outward and upward movement of the arms changes the angle that the terminal, free portion thereof, makes with the ventricular roof, such that the forces exerted on the ventricular tissue are along the axial direction of said terminal portion (thereby preventing the buckling of the anchoring arm which may otherwise occur if the anchoring arm would meet the ventricular roof at 90 degrees to said axial direction).

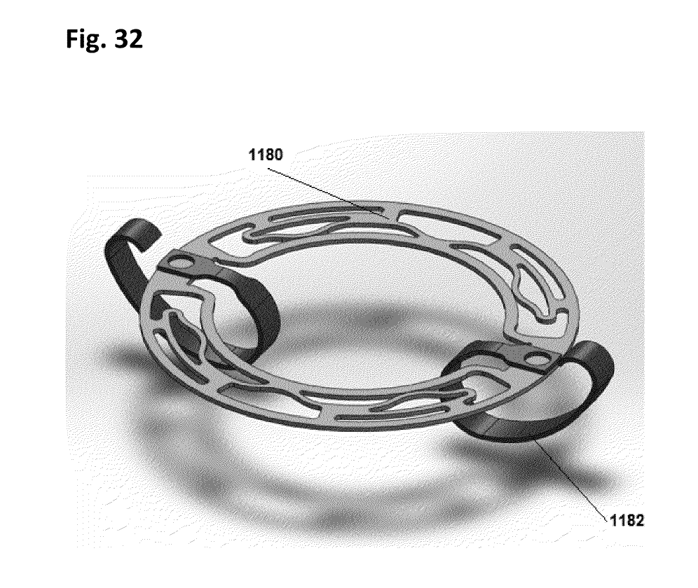

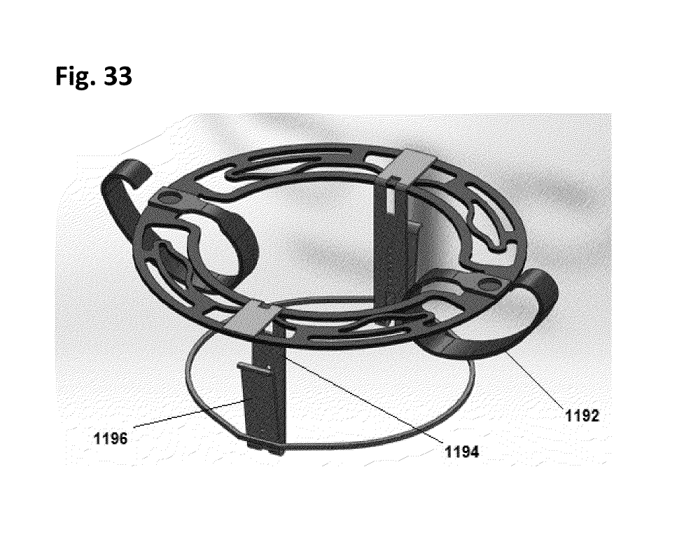

An example of this implementation of the present invention is depicted in FIG. 32. The medial ends of the curved anchoring arms 1182 are attached to the support ring 1180, while the lateral ends of said arms are seen to curve outwards and upwards. The device depicted in this figure is in its expanded state (i.e. following expansion of the replacement valve which would be placed within the central cavity of the support device), and the lateral ends of anchoring arms 1182 are shown as if they are in a plane above the plane of support ring 1180. However, in reality, said lateral ends would in fact come to rest in approximately the same plane as the support ring, and would apply strong stabilizing forces to the tissues of the ventricular roof. FIG. 33 shows the same implementation after

It should be noted that the third implementation of the device of the present invention does not utilize levers in order to obtain a force amplification effect.

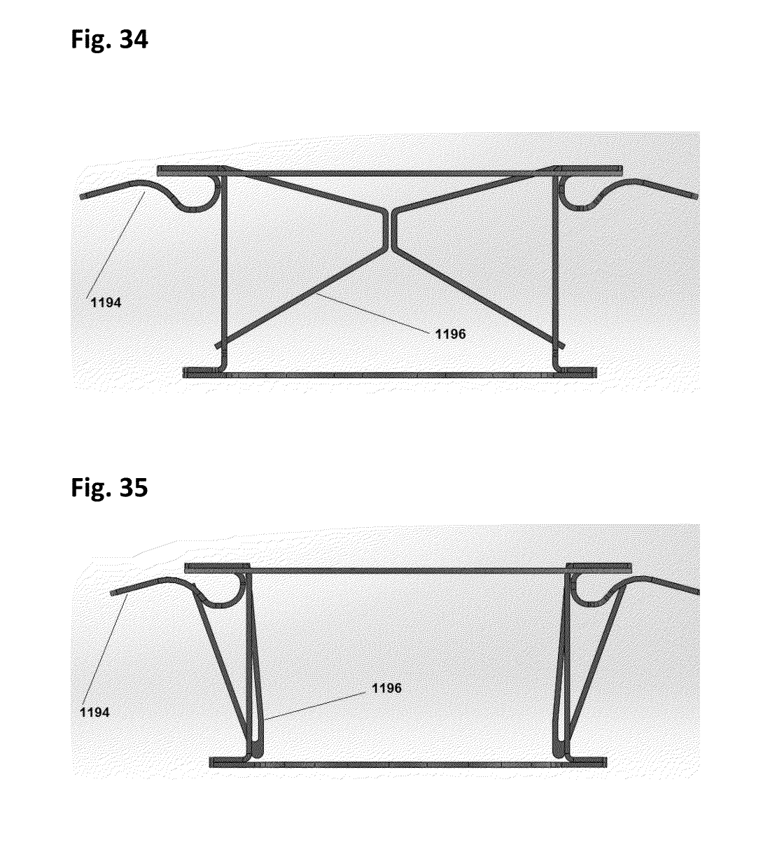

As an alternative to the third implementation of the present invention, it is also possible to construct a device comprising a combination of the first or second implementations with shorter, static anchoring arms. In such a device, the fixed arms will be used to apply relatively weak forces to the ventricular wall during the first stage of the two-stage implantation procedure, while the longer levered anchoring arms will be used to apply the stronger stabilizing forces that are required during the second stage of the procedure. In other embodiments comprising a combination of the first or second implementations with short static arms, the various anchoring elements may be arranged such that the levered anchoring arms (first or second implementations) contact the static arms (rather than ventricular tissue) during the replacement valve expansion step, thereby applying their strong stabilizing forces indirectly to the ventricular wall, that is, via the short static arms. An example of this embodiment can be seen in FIGS. 34 and 35, wherein FIG. 34 illustrates the device of the invention prior to expansion of replacement valve and FIG. 35 illustrates the device after the expansion of the replacement valve. In both figures the static arms are shown as 1194 and the levered arms are shown as 1196.

In certain other embodiments, the device may also comprise a combination of the anchoring mechanisms of several different of the above-described implementations, for example, the curved anchoring arms of the third implementation, together with the ‘leaflet pinching’ embodiment of the second implementation. Such an embodiment is shown in its expanded conformation, in perspective view, in FIG. 33. In use, the pair of curved anchoring arms 1192 will be placed along the commissural line of the native mitral valve, while the medial 1194 and lateral 1196 portions of the levered anchoring arms will be in a position such that they can be used to entrap the native mitral valve leaflets therebetween.

In certain other embodiments of the first and second implementations of the present invention, the anchoring arms and/or bridging elements (first implementation) may additionally comprise a mechanism for locking the anchoring arms in their laterally-expanded position, such that they do not apply medially-directed forces on the replacement valve. In such an embodiment, the locking mechanism may be provided by a pin connected to the bridging element, said pin being capable of interacting with an appropriately-sized aperture formed within the levered anchoring arm.

All of the components of the various embodiments of the device fitted with lever-operated stabilizing arms disclosed and described hereinabove may be constructed using any suitable biocompatible material possessing shape memory and/or superelastic properties. These properties are required in order to permit the valve support device of the invention to be transformed between a collapsed conformation (such that said device may be loaded into a delivery catheter) and an expanded, working conformation. While a preferred material for use in constructing the device is Nitinol, other suitable metallic and non-metallic materials may also be used and are included within the scope of the present invention. The various embodiments described herein may be constructed using any of the standard manufacturing techniques known to the skilled artisan in this field, including laser cutting, spot welding and so on.

In some embodiments of the present invention, the careful selection of a correctly-sized valve support device will permit said support device to be self retaining in the region of the annulus following self-expansion during device delivery, as will be described hereinbelow. In other cases, however, the valve support device of the present invention will further comprise one or more heart tissue anchoring means or mechanisms connected to the support element for firmly anchoring said valve support to the cardiac tissue. In one embodiment of this aspect, the cardiac anchoring means comprise a plurality of spiral or hook-like anchors. An example of this type of anchoring means is illustrated in FIG. 1, which shows a guide catheter 16 being used to deliver a valve support device 10 of the present invention. At the stage of the delivery process shown in this figure, the support element 12 has self-expanded into their working conformations. It will be seen that the support element is fitted with two spiral cardiac attachment anchors 18, the sharp free ends of which face laterally. The bases (i.e. medial ends) of the anchors are connected to control wires 19 that pass upwards and proximally through guide catheter 16, eventually leaving the patient's body and ending at a proximal control console. Once the valve support device has been manipulated into the desired position (as shown in the figure), the spiral anchors 18 are caused to rotate by means of the operator manipulating the proximal ends of the control wires, thereby becoming inserted within the cardiac tissue and thus firmly anchoring the valve support device in its operating position.

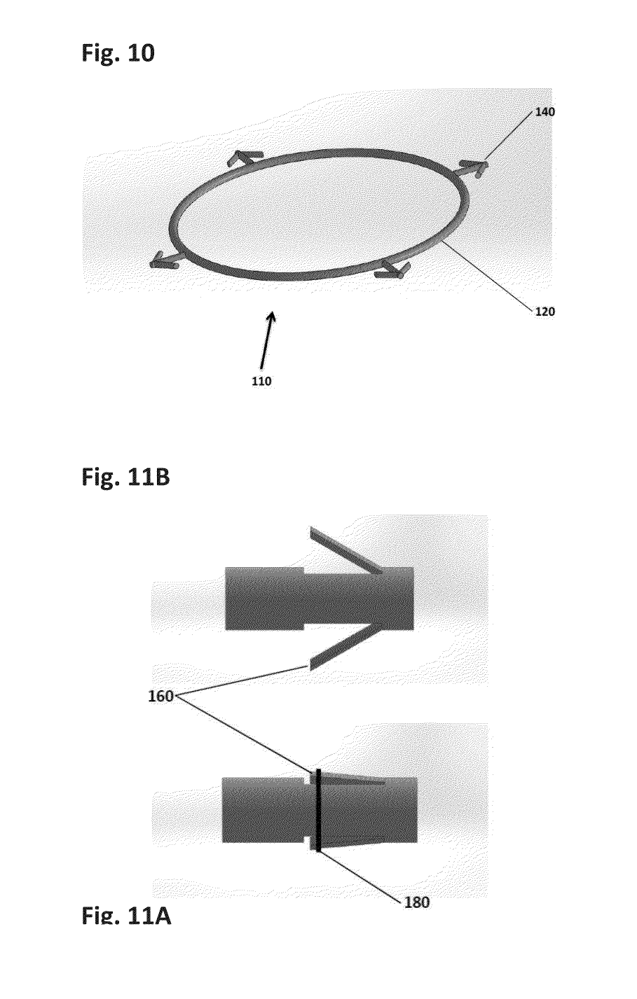

It is to be noted that FIG. 1 presents only one exemplary design for the cardiac tissue anchors, and many others are possible and included within the scope of the present invention. Thus, in another embodiment, hook-like anchors are attached at various points along the surface of the valve support device. This embodiment is illustrated in FIG. 10 which depicts a typical valve support device 110, comprising a support element 120 on the surface of which are distributed a number of hook-like anchors 140. (Four such anchors are shown in the figure.)

In some situations, it is advantageous for the cardiac tissue anchors to adopt a closed, inactive conformation during insertion of the valve support device into the body, in order to avoid both trauma to the patients tissues and to avoid premature anchoring (for example at an incorrect location). Then, when said device is correctly positioned, the anchors would be caused to move from their closed, inactive conformation to an open active position. There are a number of ways to implement this type of embodiment. Thus, in a first implementation, the cardiac attachment anchor is constructed with two or more backwardly-pointing self-opening distal arms. During insertion and implantation, the distal arms are retained in a closed conformation by means of a small loop of resorbable suture material. Then, after a certain period of time following insertion of said attachment means into the ventricular tissue (e.g. between a few hours and a few weeks), said suture dissolves, thereby permitting the distal arms to adopt their open conformation. This embodiment is illustrated in FIGS. 11a and 11b: in FIG. 11a, the distal anchor arms 160 are shown retained in their closed position by means of suture 180. In FIG. 11b, the required length of time has elapsed (following insertion) and the suture has dissolved, releasing the distal anchor arms and allowing them to spread apart within the cardiac tissue, thereby increasing the resistance to withdrawal offered by said anchor.

In a further embodiment of this type, the anchor hooks are manufactured from a shape memory material, such as biocompatible nickel-titanium alloys (e.g. Nitinol). During insertion, the anchors are in their closed conformation, but following the implantation procedure the rise in temperature experienced during insertion into the patient's body results in opening of the anchors, as they regain their initial shape.

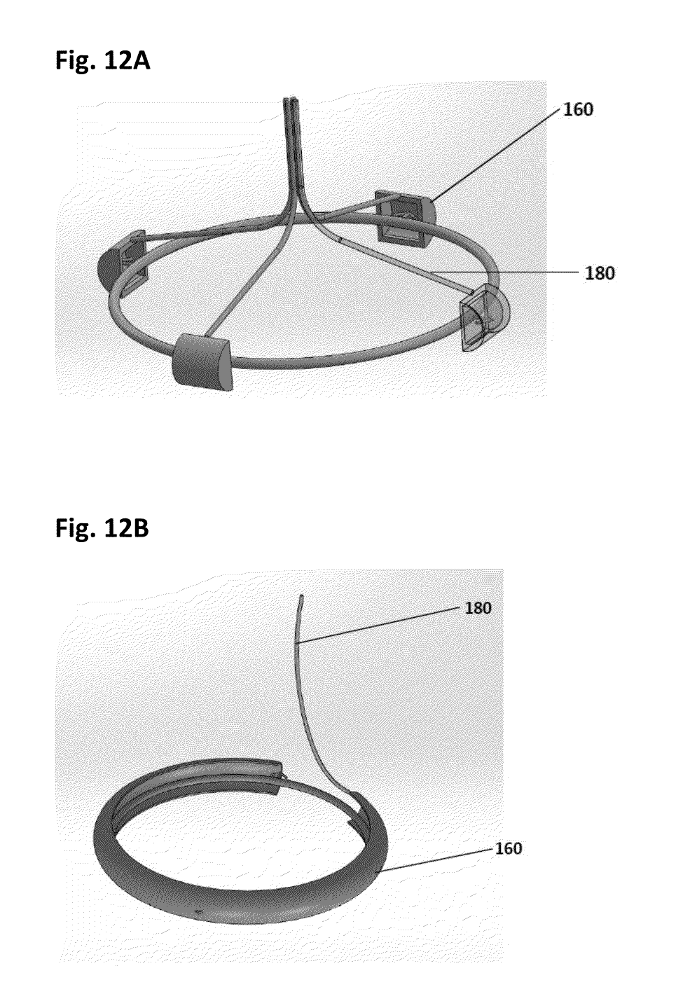

In a still further embodiment of this type, as shown in FIGS. 12A and 12B, the anchor hooks are protected by a cover element 160 (such as a sleeve or a piece of tubing) which is manufactured from a material with limited flexibility, such as PET, nylon and similar biocompatible plastics. After the operator is satisfied that the valve support device has been implanted at the correct site, control elements 180 (e.g. wired) attached to the cover elements are pulled, thereby withdrawing them through the guide catheter, thus permitting the anchor hooks to freely adopt their open conformation and to become inserted into the cardiac tissue. In the design shown in FIG. 12A, each anchor is protected by its own individual cover, while in FIG. 12B a single cover element protects all of the anchors (not shown) that are attached to the support element.

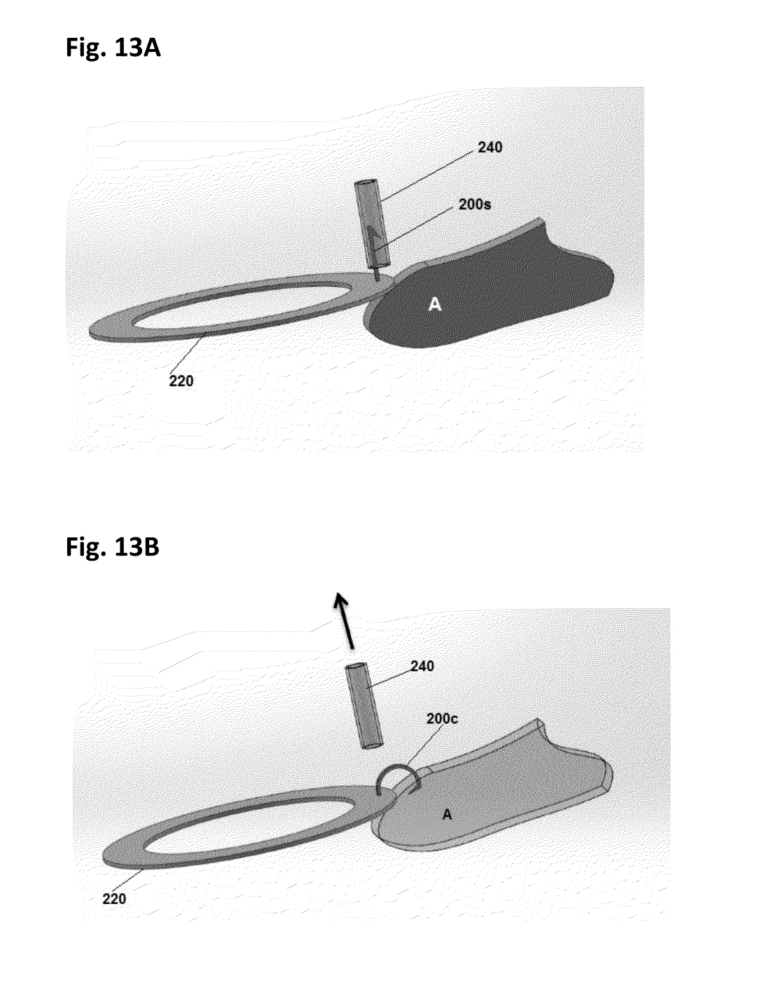

FIGS. 13A and 13B illustrate a yet further embodiment of this aspect of the invention. Thus FIG. 13A shows a barbed anchor 200s attached to a support element 220 is maintained in an inactive, straight conformation by means of an overtube 240, which also serves to protect the patient's tissues from trauma during insertion and implantation of the valve support device. Following implantation at the desired site, as shown in FIG. 20B, overtube 240 is pulled away from the anchor 200c (for example, by means of pulling a control wire), which now adopts its “natural”, curved conformation, during which shape transition, said anchor now pierces the cardiac tissue (indicated by the letter A in the figure). Suitable anchors for use in this embodiment can be manufactured from shape-memory materials or from super-elasticity materials such as Nitinol, cobalt base alloy and spring-tempered stainless steel. Typically, anchors of this type will have a mid-length diameter of between about 0.2 mm and 1 mm, and a length in the range of about 2 to about 10 mm. Suitable overtubes may be manufactured from biocompatible polymers such as braided nylon and PET to a tolerance that permits a tight fit over the anchor.

It is to be noted that the cardiac tissue anchors described hereinabove may, in certain cases, be used to attach the valve support device of the present invention to the anatomical valve leaflets and chordae (in addition to, or instead of attaching said device to the inner ventricular wall). In this regard, the present invention also encompasses additional types of cardiac tissue anchor which are characterized by having a plurality of anchoring wires that advantageously become entangled within the valve leaflets and chordae. Anchors of this type are particularly suitable for use in attaching the support element to the aforementioned anatomical structures.

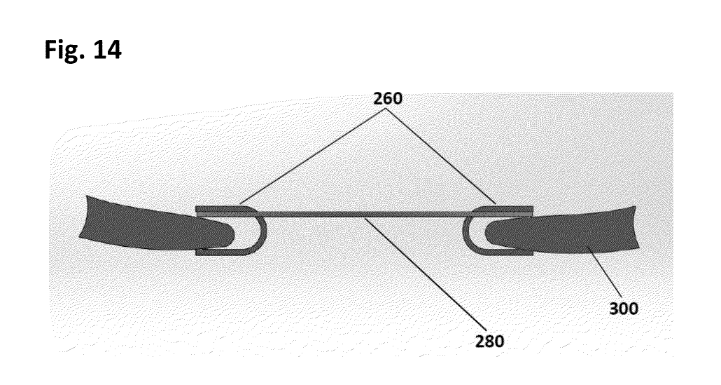

In one still further embodiment, the cardiac tissue anchors may be provided in the form of small clips (similar to vascular clips used to close blood vessels during surgical procedures, and well known to the skilled artisan). An example of the use of this embodiment is shown in FIG. 14, in which clip 260 is used to attach the support element 280 to the annulus 300. Clips of this type may also be used to attach the support element to atrial wall tissue and/or anatomical valve leaflets. In one particularly preferred embodiment the clip is caused to attach to the tissue in the area of the trigone—an anatomical area, on two opposite sides of the mitral valve, which has more fibrous tissue—and which is therefore able to provide a firm base for anchoring the valve support device.

In another embodiment (not shown), the clip may be an integral part of the support element. This may be achieved by attaching one of the jaws of the clip to the valve support device, while the second of the jaws is free to be plastically deformed and to become anchored to the tissue.

In the case of certain replacement valves that may be used in conjunction with the valve support device of the present invention, the radially-outward forces exerted by the expanded replacement valve are sufficient to stably retain said valve within the inner cavity of said valve support device. However, in some instances—particularly when self-expanding replacement valves are being implanted—the radial force exerted by the expanded valve may be insufficient to ensure that it can withstand all of the physiological forces exerted therein during all stages of the cardiac cycle. In such circumstances, the single-ring support element of the valve support device may further comprise a valve engagement portion. In one embodiment, said valve engagement means comprise either inward facing or outward facing anchors, whose purpose is engage with the external struts of the replacement valve, thereby stabilizing said valve within the support device.

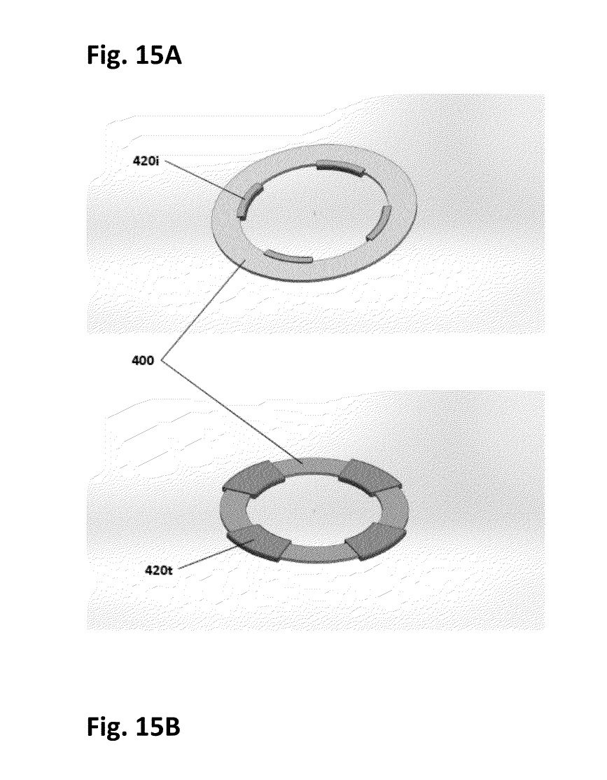

FIGS. 15A and 15B show a further embodiment of the valve engagement means, attached to an exemplary support element 400 of the present invention. Thus, in FIG. 15A, four short lengths of a soft biocompatible material (such as a biocompatible fabric, silicon, PET etc.) 420i are attached to the inner surface of support element 400. Upon expansion of the replacement valve stent within the inner space of the valve support device, the soft material is caused to penetrate between the valve stent struts, thereby forming engagement “teeth” that serve to stabilize the replacement valve—support device assembly. FIG. 15B depicts a very similar set of four valve engagement means 420t formed from a soft biocompatible material. However, in the case of this version, the soft material is provided in the form of tubular sleeves surrounding (partially or completely) support element 400 at the four locations shown in the figure.

As disclosed hereinabove, in certain embodiments, the valve support device of the present invention further comprises one or more height-increasing elements attached to the inner circumference of the annular support ring. The advantages of the height-increasing elements stem from the fact that in essence they transform the flat ring shape into a tube-like shape, thus better mimicking the shape into which the said prosthetic valves were originally designed for (since both the aortic valve and the aorta have an essentially tubular shape).

In one preferred embodiment of this aspect of the invention, the aforementioned height-increasing element is provided by a wire spring, manufactured from a biocompatible metal such as Nitinol. In this embodiment, the lower border of said spring may be attached to the inner circumference of the support ring(s) such that said spring is located above the plane of said ring(s) (i.e. towards the atrium). In an alternative version of this embodiment, the upper border of said spring may be attached to the inner circumference of the support ring(s), such that said spring is located below the plane of said ring(s) (i.e. towards the ventricle). In a yet further version of this embodiment, the central portion of the spring is attached to the inner circumference of the support ring(s), such that portions of said spring are disposed both above and below the plane of the support ring(s). Finally, in another version, two separate springs may be present, with one spring disposed above the plane of the support ring, and the second spring disposed below this plane. The spring elements may be manufactured separately from the device, and then attached thereto at the desired location with the aid of surgical sutures or other mechanical form of attachment, within a biocompatible fabric covering the device. Furthermore, in some embodiments, said spring elements may be covered with biocompatible plastic or polymer, for example a silicone tube.

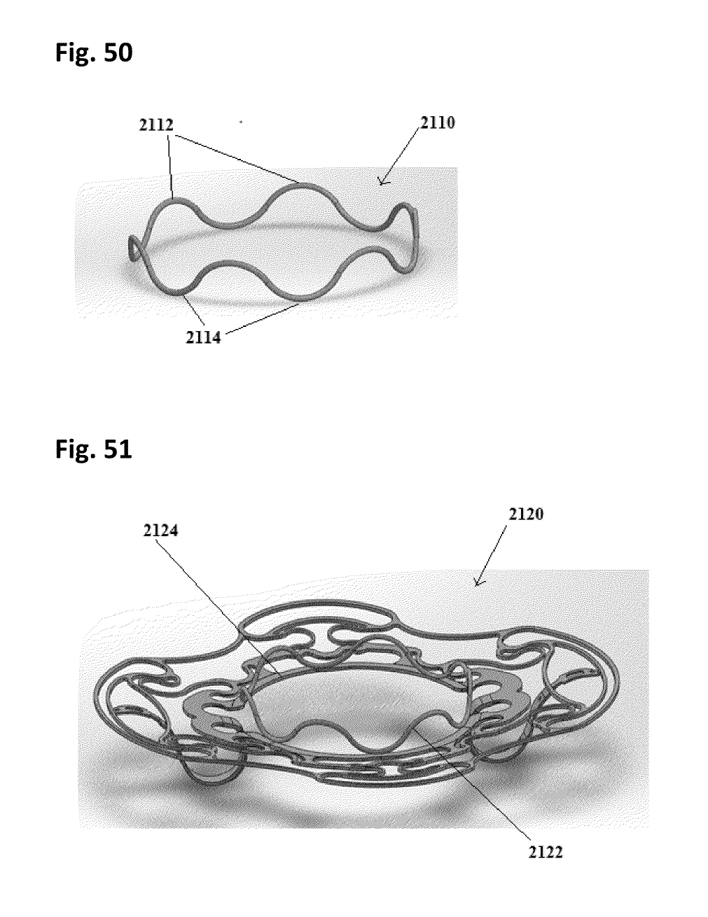

In one preferred version of this embodiment of the invention, the wire spring is constructed in the form of a wavy wire (for example, having the approximate shape of a sinusoidal wave) having its two ends joined together, thereby causing said spring to adopt the approximate shape and form of a closed loop (similar to a common type of bed-spring). An example of such a wavy spring loop is depicted in FIG. 50. In this figure, spring 2110 is seen to comprise an upper border (formed by an imaginary line joining the highest portions 2112 of said spring, and a lower border, formed by an imaginary line joining the lowest portions 2114 thereof.

FIG. 51, depicts a single-ring valve support device 2120 in which a single wavy spring loop 2122 (as shown in FIG. 50) is attached at its lower border to the inner circumference of support ring 2124. As may be seen in this figure, this type of attachment leads to an increase in the effective height of the support ring in an upwards direction (i.e. towards the atrium).

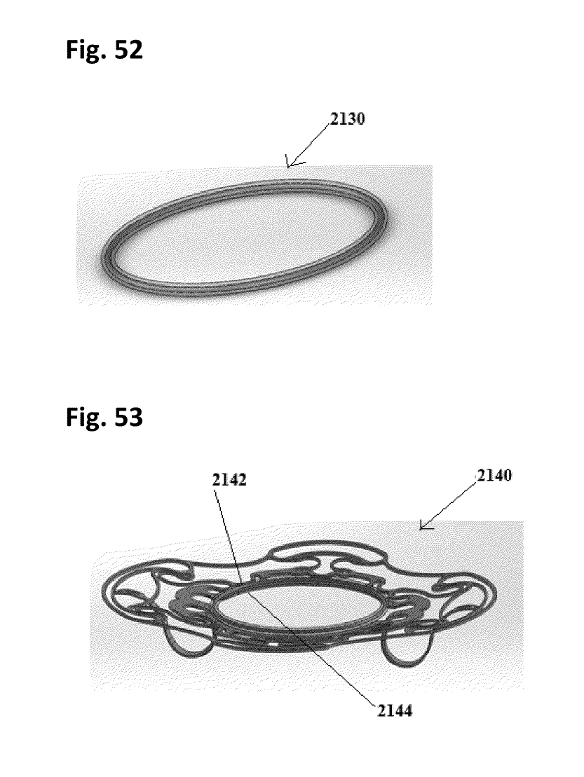

In one preferred version of this embodiment of the invention, the wire spring is constructed in the form of a non-wavy, hoop-like circle, which may be attached either to the upper face of the valve support ring, adjacent to the inner circumference thereof (thereby increasing the height of said ring in an upward direction), or to the lower face thereof (thereby increasing the height of said ring in a lower direction). Two or more such wires may be used to increase the height of said ring even further, and may be attached to the upper face of the valve support ring, the lower face, or both. An example of this type of wire hoop spring is shown (2130) in FIG. 52.

FIG. 53 illustrates a single-ring valve support device 2140, in which a wire hoop spring 2142 (of the same design as shown in FIG. 52) is attached to the upper face of support ring 2144, close to the inner circumference thereof.

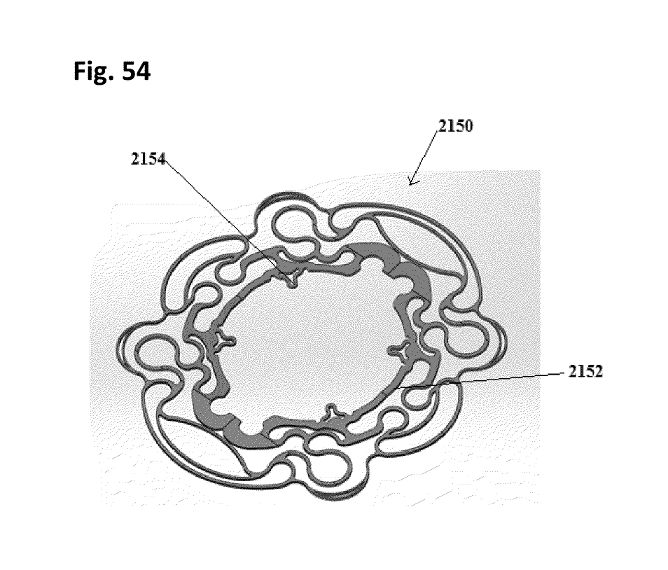

In a further embodiment of this aspect of the present invention, the height-increasing elements are provided in the form of tab-like elements formed as part of the inner circumference of the one or more support rings, wherein said tab-like elements are caused to fold upwards, downwards (or both upwards and downwards) in relation to the plane of the support ring(s), thereby effectively increasing the vertical height of said ring(s) in these directions. The invention may comprise any suitable number of tabs or tab-like elements, preferably two or more. FIG. 54 illustrates a single-ring valve support device 2150 of the present invention in its flat, pre-folded form, in which the inner circumference of the support ring 2152 includes four tab-like elements 2154. As part of the stage of manufacturing and shaping of this flat-form of the device, the tab-like elements will be caused to be folded and fixed (by means of heat setting) in the desired directions (upwards, downwards, or both).

In all embodiments of this aspect of the present invention, the entire device (including the said additional elements above, below, or both above and below the inner border of the support ring) may be covered with a fabric (such as PTFE, Dacron, Polyester or other biocompatible material). When covered in this way, the shape of the internal border of the support ring is essentially transferred from being a flat ring shape to a 3 dimensional tube-like element, having a longitudinal length (height) of approximately 3-20 mm, and a thickness of 0.2-1 mm.

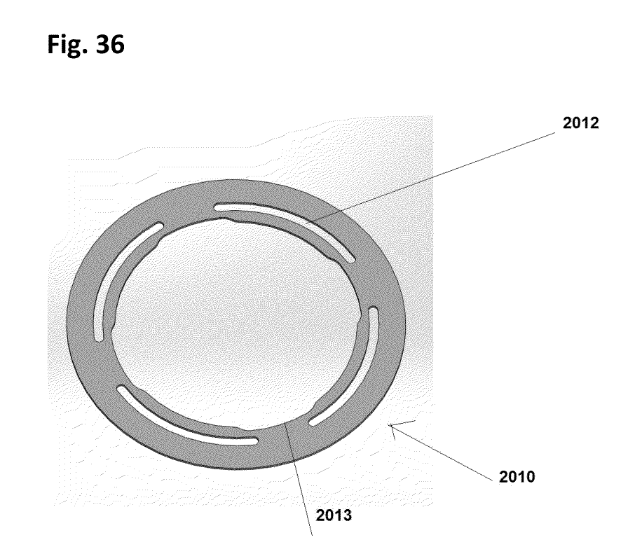

As explained hereinabove, in certain preferred embodiments of the present invention, the inner perimeter of the annular single-ring support element is able to elastically deform in a radial direction, in order to enhance the stability of the replacement valve within the central space of the valve support device. Thus, in the preferred embodiment illustrated in FIG. 36 the support element 1210 includes cut-out areas 1212, which are cut out from the central area of the element (from the “body” of the ring), and cut-out areas 1213 which are cut out from the inner part of the element (from the internal perimeter of the ring). In this example, there are four such 1212 cut-out areas and 4 such 1213 cut-out areas. The number and shape of these cut-out areas is exemplary only, and any number and shapes may be used. An exemplary material for manufacturing the support element is biocompatible metal or alloy (for example Nitinol or stainless steel). The goal of both cut out areas 1212 and 1213 is to make the support element elastically deformable at the inner perimeter of the ring, to enable radially inward forces to be applied when a stented valve is expanded within the support element.

Exemplary sizes for the device of the invention: for example, the internal diameter of the support element 1210 in a “resting” state (the baseline stent, after the ring is deployed in the Mitral annulus, but before a stented valve is deployed and expanded within the ring) may be 25 mm. An exemplary 26 mm diameter valve (e.g. the Sapien valve manufactured by Edwards Lifesciences Inc., USA) is now expanded within the support element by means of balloon expansion to a diameter of 27 mm, and immediately after expansion it has some recoil to a diameter of 26 mm. Since the valve was expanded within the support element, the internal ring diameter is now (after expansion) directly approximated to the valve, so the inner diameter of the support element ring is now 26 mm. Since, as said in the example, the resting diameter of the support element is 25 mm, than due to the elastic ability of the support element in the design of this invention, the support element now applies a radially inward force on the valve, and thus is strongly secured to the valve and prevents the valve from dislocating. Of course, sizing may change according to the desired valve, and this is an example only.

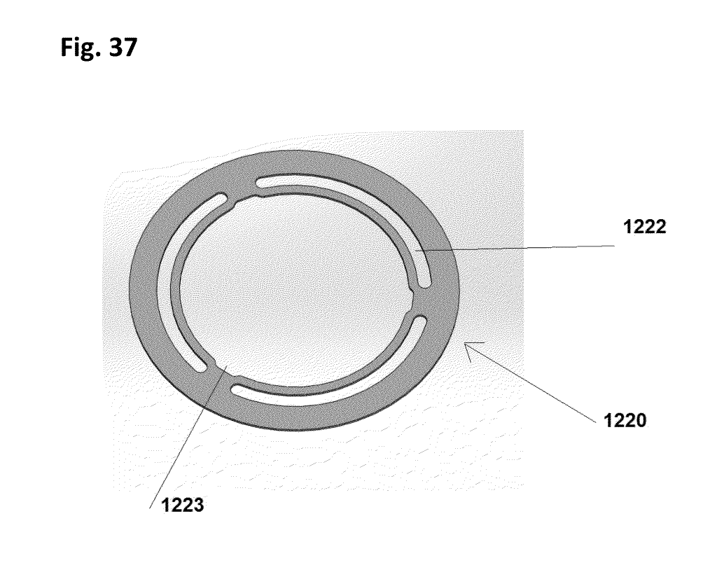

FIG. 37 illustrates another exemplary embodiment of a single-ring support element of a valve support device of the present invention. In the preferred embodiment illustrated in this figure, the support element 1220 includes three cut-out areas 1222, which are cut out from the central area of the element (from the “body” of the ring), and three cut-out areas 1223 which are cut out from the inner part of the element (from the internal perimeter of the ring). The number and shape of these cut-out areas is exemplary only, and any number and shapes may be used.

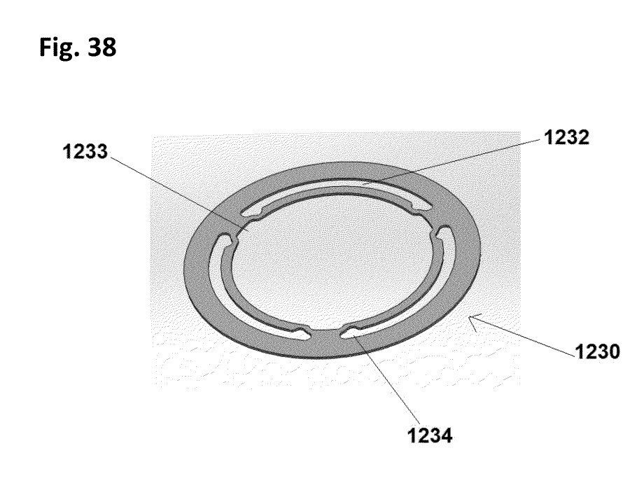

FIG. 38 illustrates a further exemplary embodiment of a single-ring support element of the present invention. In the preferred embodiment illustrated in this figure, the support element 1230 includes three cut-out areas 1232, which are cut out from the central area of the element (from the “body” of the ring), and three cut-out areas 1233 which are cut out from the inner part of the element (from the internal perimeter of the ring). The number and shape of these cut-out areas is exemplary only, and any number and shapes may be used. In this embodiment, each cut-out area 1232 includes additional cut out areas 1234 which modify and increase the elasticity of the support element. Any number or shapes of such additional cut-out areas are within the scope of the present invention.

As explained hereinabove, in certain preferred embodiments of the present invention, the single-ring support device comprises reduced-diameter, cut-out regions in its external perimeter, the purpose of which is to act as a pressure-release element, thereby permitting the controlled, limited regurgitation of the cardiac valve, as a means of reducing the overall fluid pressure exerted on the cardiac valve support device and prosthetic valve. In this way, the stability of the implanted prosthetic valve may be improved. Thus it may be appreciated that the pressure-release feature of this aspect of the invention reduces the total fluid pressure applied on the valve support—replacement valve apparatus by the contracting heart, thus reducing the upward forces applied on said apparatus. Additionally, this design reduces the afterload against which the ventricle contracts, since it allows a controlled limited regurgitation, and thus may have beneficial clinical effects on ventricular function. In this design, the shape of the single-ring support structure does not completely cover the shape of the annulus, and does not have a complete circular shape, but rather has an outline shape that imparts the following two advantages: 1—A part of the single-ring support structure has a larger diameter than the annulus diameter (i.e. an expanded diameter segment), thus when the support structure is expanded above the annulus—the larger diameter of the shape prevents it from “falling down” across the annulus from the atrium into the left ventricle, and thus assists in maintaining the valve support in its intended location in the mitral annulus. 2—One or more parts of the single-ring support structure have a smaller diameter than the annulus diameter (i.e. a reduced diameter segment), thus when the support structure is expanded above the annulus, there are one or more apertures that remain “open” between the atrium and the ventricle. This actually causes a leak, or essentially a controlled “MR” (Mitral Regurgitation), the magnitude of which is predetermined by the size and number of the apertures.

Clinical theoretical explanation: It is pertinent at this point to explain how the intentional production of a “controlled MR” may be clinically valuable for a patient who is being treated with valve-replacement to correct his pre-existing MR. Thus, patients undergoing valve replacement for MR usually suffer from grade 3 or 4 MR, which results in significant clinical symptoms thereby necessitating clinical intervention. Optimally, the goal is to replace the valve and reach zero MR (no leak). However, it is clinically acceptable to complete a procedure such that the patient remains with a small residual MR (grade 1), since it would still be significantly better than stage 4 before the procedure, and since the device of this invention allows a trans-catheter implantation instead of surgery for valve replacement, the “cost” in outcome would be stage 1 MR (with a minimally-invasive procedure) instead of zero MR (using a surgical approach), this would be clinically beneficial for some patients, especially those having co-morbidities associated with a very high surgical risk.

An additional advantage of this embodiment, in which the apertures between each of the reduced diameter segments and the adjacent portion of the annulus permit the limited peri-valvular flow of blood between the ventricle and the atrium, is that after implantation of the valve support there is maintained a “controlled” or “limited” amount of regurgitation (flow during systole from the ventricle into the atria through the perivalvular apertures). This reduces the afterload, the force against which the left ventricle (LV) contracts, and may be advantageous in cases of reduced systolic performance of the left ventricle. Such afterload reduction may potentially be beneficial to improve left ventricular performance, reduce LV wall stress and oxygen consumption.

Sizing example and explanation: The following sizes are exemplary only, and are provided in order to illustrate the principle on which this embodiment of the present invention is based.

For an exemplary mitral annulus diameter of 35 mm. The inner diameter of the single-ring support element has to be appropriate for the expanded diameter of the stented replacement valve which is to be expanded in the valve support. For an exemplary Sapien 26 mm valve, the inner diameter of the upper ring is approximately 26 mm. The outer diameter of the support element should be larger than the annulus diameter, in order to prevent the device from “falling” into the ventricle, and in order to assist in prevention of para-valvular leak. Hence for this example an outer diameter of 37 mm is chosen. However, at least one part of the support ring will have a diameter which is smaller than 35 mm (for example a cut out will be made in a part of the outer perimeter of the ring, thereby reducing the local diameter to only 33 mm), thus causing a small aperture between the outer edge of the upper ring and the mitral annulus. During systolic ventricular contraction these one or more apertures function as a pressure release mechanism—they release some of the pressure (upward force) applied on the valve support-valve apparatus, and thus reduce the risk that the apparatus will be dislocated out of position.

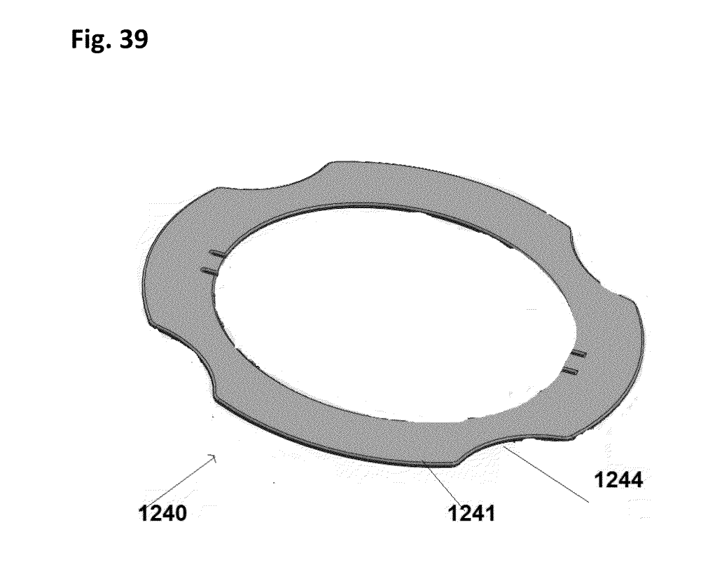

Thus, FIG. 39 provides a perspective view of an exemplary embodiment of a valve support of this embodiment of the present invention in an expanded configuration. Valve support 1240 includes an annular single-ring support element 1241, having four areas of reduced diameter (cut-out areas from the perimeter of the ring), 1244. This number of such smaller diameter areas 1244, as well as their size and shape, are given by way of example only.

In another preferred embodiment, as explained hereinabove, the annular support element is fitted with least one wing-like drape element made of a biocompatible fabric. The drape functions as a sealing element, positioned between the support element and the mitral annulus, thereby preventing paravalvular leakage (PVL) after implantation of the valve support in the mitral annulus.

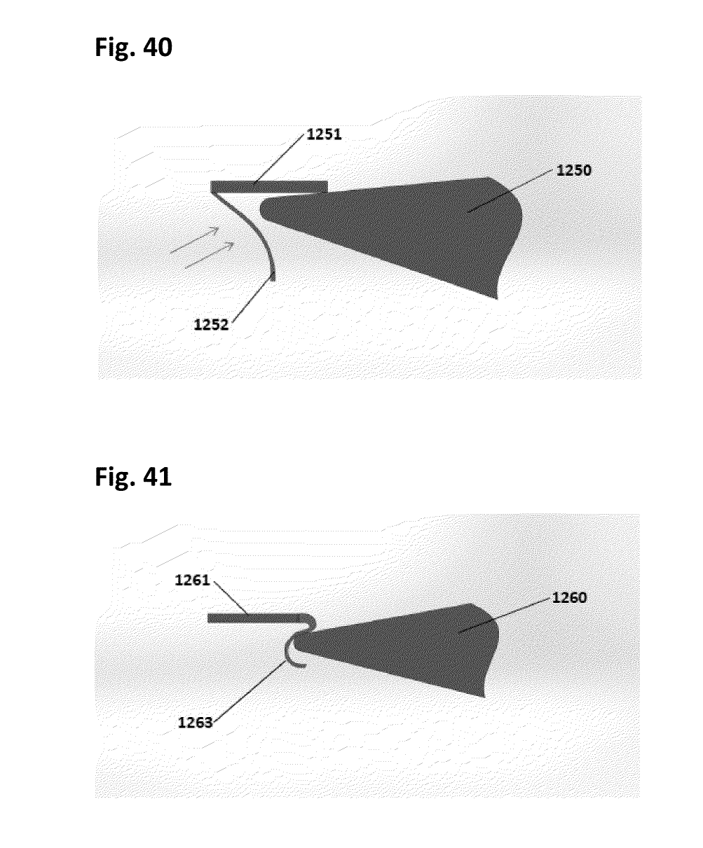

Thus, FIG. 40 illustrates a side view of an exemplary single-ring support device 1251 of the present invention, shown in position on the mitral annulus 1250, and comprising a fabric drape 1252 attached to the inner circumference of the ring. The position of the drape on the inner circumference of the ring support presents several distinctive advantages: the drape functions as a “valve leaflet” between the mitral annulus and the upper ring, thus during systole, when fluid flows out from the ventricle, the drape is pushed upwards, towards the annulus, by the flow of blood, and this movement improves the sealing between the upper ring and the annulus, (indicated by the arrows in FIG. 40), thus essentially functioning as a valve between the ring and the annulus, and thus preventing PVL.

FIG. 41 illustrates a side view of an exemplary single-ring support device 1261 of the present invention, shown in position on the mitral annulus 1260, with a fabric drape 1263 attached to the outer circumference of said ring. The position of the drape on the outer part of the ring support allows it to function as a sealing element between the ring and the annulus (similar to the function of a sealing “o” ring), so when the ring is approximated and attached to the area of the annulus the drape functions to seal the annulus and prevents PVL. In some embodiments of this invention the length of the drape is such that the edge of the drape extends into the left ventricle (as shown in FIG. 41). This is advantageous since this extended drape element improves the sealing and prevents leakage between the outer area of the ring and the mitral annulus.

Exemplary materials for the drape of the invention are any kind of biocompatible fabric, for example Dacron, ePTFE. Exemplary sized of the drapes of the invention are length of 2 mm-20 mm and width of 2 mm-60 mm, thus covering a part of the ring or the whole circumference of the ring.

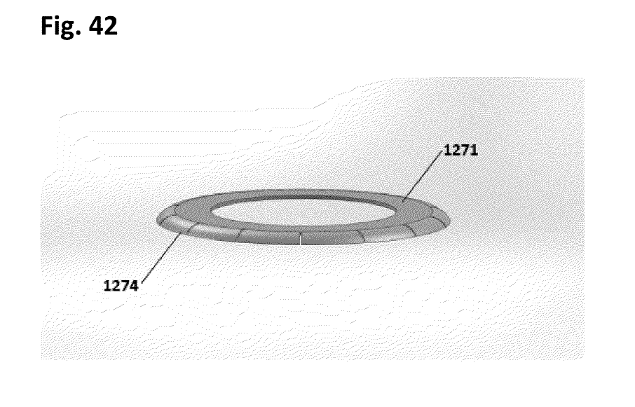

FIG. 42 illustrates a perspective view of an exemplary single-ring support device 1271 of the present invention, exemplifying multiple fabric drapes 1274. Five such separate drapes are shown, with the rest of the drapes not shown in the illustration. Preferably there is a small overlap between drapes, such that there is no leakage between adjacent drapes.

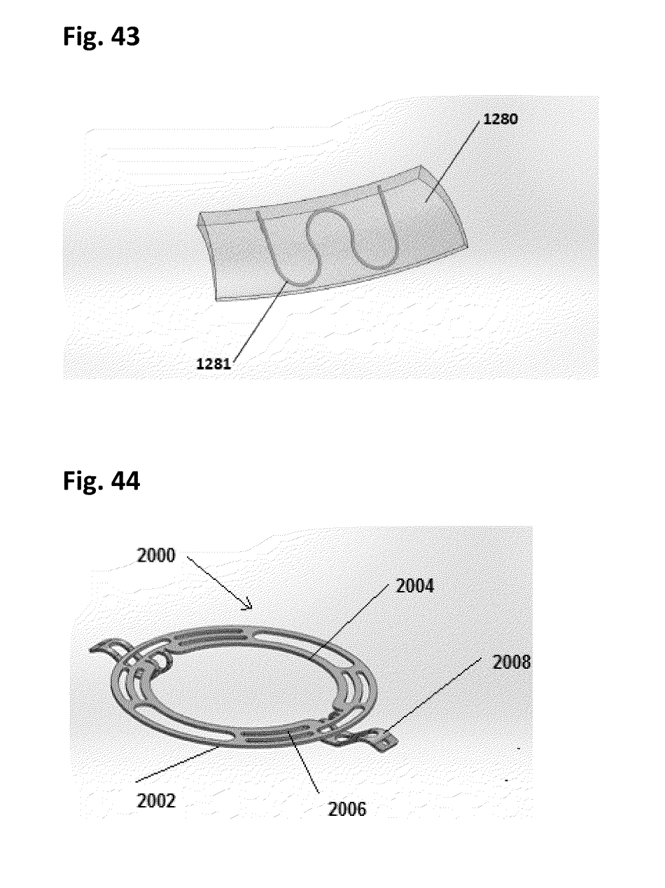

FIG. 43 illustrates a further design for the fabric of a drape of the invention. Drape 1280 as shown in this figure is made of biocompatible fabric. In order to give the drape a stable form (in order that it will have a predetermined shape), a biocompatible metal wire 1281 is sewn into the material of the drape during its manufacturing. Exemplary materials for the wire are stainless steel or Nitinol. The metal wire can be shaped according to a predetermined requirement, and is able to maintain this shape due to the mechanical properties of the wire. The advantage of this predetermined shaping is that the shape may be designed such that it will improve the sealing between the ring and the annulus, so that the flow will direct the drape towards sealing the annulus, moving the drape closer to the annulus thereby preventing PVL.

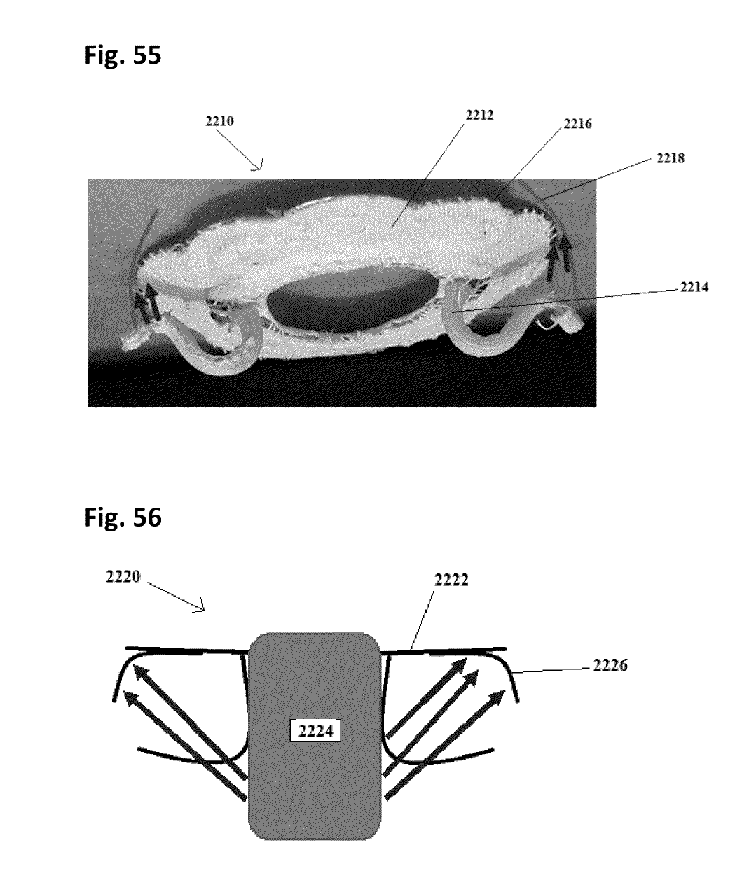

As explained hereinabove, in certain preferred embodiments, the annular support element of the present invention may be fitted with other means for reducing PVL, wherein said means are selected from the group consisting of: lateral edge extensions, one or more tubular sealing elements, one or more barbs, an inferiorly-directed circumferential fabric skirt attached to the inner circumference of the ring-like valve support device and an inferiorly-directed fabric curtain attached to the outer circumference of said device. In certain circumstances, significant PVL can occur between a valve support device implanted within a valve annulus and the adjacent cardiac tissues. This state of affairs is shown in FIG. 55 which depicts a mitral valve support device 2210 of the present invention comprising a single support ring 2212 and two stabilizing wings 2214. Curved lines 2218 are intended to show the position of the atrial wall in relation to support device 2210, after said device has been implanted at the mitral annulus. The pairs of upwardly-pointing arrows indicate the position and direction of the PVL, that is, into the pocket-like region where the outer circumference of the annular support device meets the atrial wall. In many cases, the leakage problem is exacerbated by the presence of a replacement valve within the central space of the annular valve support device, said valve causing the lateral displacement of the native valve leaflets and altering the topology of the region of the annulus adjacent to the implanted support ring.

The present inventors have found that it is possible to overcome this leakage problem by means of altering the shape of the lateral portion of the valve support ring. In one embodiment, this may be achieved by the presence of an additional lateral extension having an origin on the support ring and a free edge that extends latero-inferiorly from said origin. In another embodiment, the outer portion of the support ring itself is caused (during manufacture) to curve laterally and inferiorly. In either case, the lateral extension or the downwardly curved outer ring portion is constructed such that it is very flexible, thereby enabling it to conform to the anatomy of the atrial wall. In one preferred embodiment, the lateral extension or curved outer ring portion is constructed from Nitinol having a thickness of 0.1-0.5 mm, preferably 0.1 or 0.2 mm.

FIG. 56 illustrates the first of the two above-mentioned embodiments, in which valve support device 2220 comprises a single support ring 2222 and a lateral extension 2226 which extends around the entire circumference of said ring. Replacement valve 2224 has been implanted within the central space of said support device. It will further be seen in this figure that the flow of blood (indicated by the diagonally-orientated arrows) in the region of said replacement valve ends at the lateral extension, which thereby prevents the leakage of blood around the lateral edge of support ring 2222. Furthermore, the fluid flow itself at this point will tend to cause a small displacement of the lateral extension in an upward and lateral direction, thereby further improving the fluid seal provided by said extension.

A further solution found by the present inventors is the use of a sealing ring attached to the outer portion of the annular valve support ring, wherein said ring may be either continuous around the entire circumference of said support ring (i.e. similar to the form of an O-ring) or may be discontinuous, consisting of discrete portions. In one preferred embodiment, the sealing ring may have different elasticity values in different location. Thus, for example, the ring may have a higher elasticity in the area of the aorta. In one preferred embodiment, said sealing ring may be constructed from a braided tube (for example made of Nitinol wires, or wires constructed from another biocompatible metal) covered with a biocompatible fabric (such as PTFE, Dacron, Polyester or other biocompatible fabrics). The braided tube may be manufactured in two steps, wherein the first step comprises braiding the said wires into a tube shape, and the second stage consists of covering the braid with biocompatible fabric. The braid may be formed into a ring shape by closing the free edges thereof. Exemplary dimensions for such braided tubes are wires having a thickness of 0.03 mm-1 mm, exemplary number of wires in the braiding may be 8-64 wires, and typically the diameter of the braided tube may be 2-15 mm.

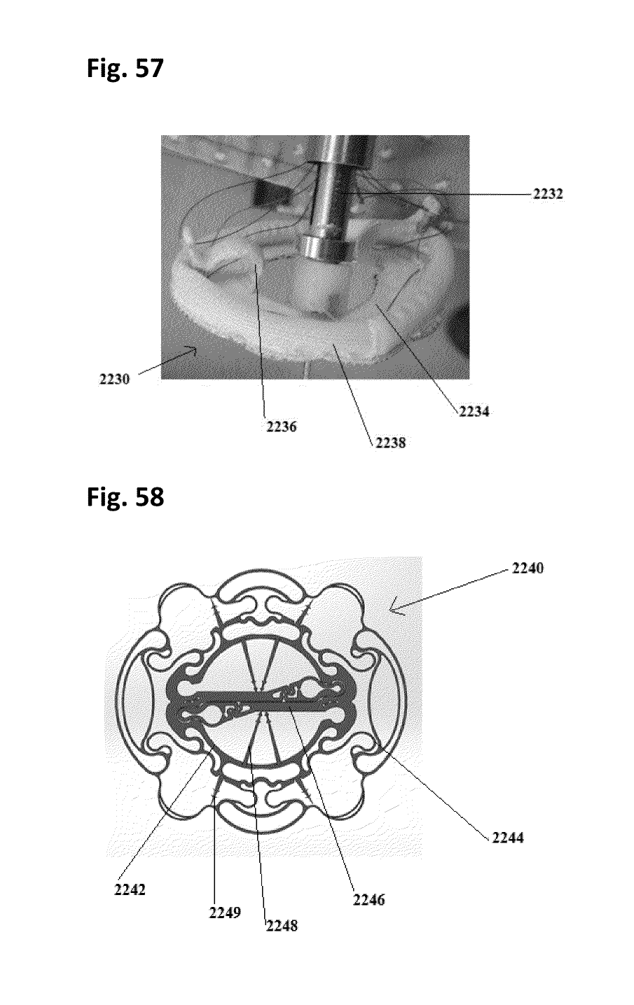

One example of this embodiment is illustrated in FIG. 57 which shows a mitral valve support device 2230 comprising a single support ring 2234 and two stabilizing or anchoring wings 2236 held within a delivery device 2232 by a series of wires. As shown in this figure, a complete sealing ring 2238 constructed from a fabric-covered braided tube is attached to the outer portion of said support ring. In this example, the wire used to manufacture the braided tube has a diameter of 0.1 mm while the tube itself has an external diameter of 4.6 mm. During use, the sealing ring will become compressed against the cardiac tissues of the atrial wall and mitral annulus, thereby ensuring complete sealing at all stages of the cardiac cycle, and thus preventing PVL. A unique feature of the braided tube of the invention is the fact that its mechanical characteristics allow it to apply forces on the cardiac tissue when the device is deployed in its working position (said forces rising from the expansion of the braid), and thus the fabric of the braid (which does not allow blood penetration) is approximated to the cardiac tissue in different anatomic positions, different anatomic sizes and shapes, and in different parts of the cardiac cycle—maintaining a constant sealing to prevent leakage. At the same time, and very importantly, the braided structure allows crimping of the valve support device to a very small size, thereby enabling transcatheter delivery of the device. By way of example, a braided sealing tube which in its “resting” state has a diameter of approximately 7 mm, braided from 42 wires which are 0.06 mm thick, can be crimped to a diameter of less 1 mm. In another embodiment of this aspect of the invention, the sealing ring is constructed from a metallic sponge-like material (e.g. a metallic wool).

A further approach that has been adopted is to use barb-like prongs attached to various portions of the annular valve support device, wherein the free ends of said prongs become embedded within the cardiac tissue, thereby improving the apposition of said device to the cardiac tissues and thus preventing or reducing PVL. FIG. 58 illustrates one version of this embodiment, a mitral valve support device 2240 comprising a single support ring 2242 connected on its lateral surface to an intricate crown-like lateral portion 2244 and two stabilizing wings 2246. (Said valve support device is depicted in this figure in flat form, after having been cut out of a Nitinol sheet, but before being crimped into a delivery device.) It will be noted that the support device shown in this figure comprises a total of eight prongs fitted with barbs—four relatively long prongs 2248 attached to the inner circumference of support ring 2242, and four shorter prongs 2249 attached to the crown-like lateral portion 2244. Devices having fewer or greater numbers of prongs are also included within the scope of the present invention.

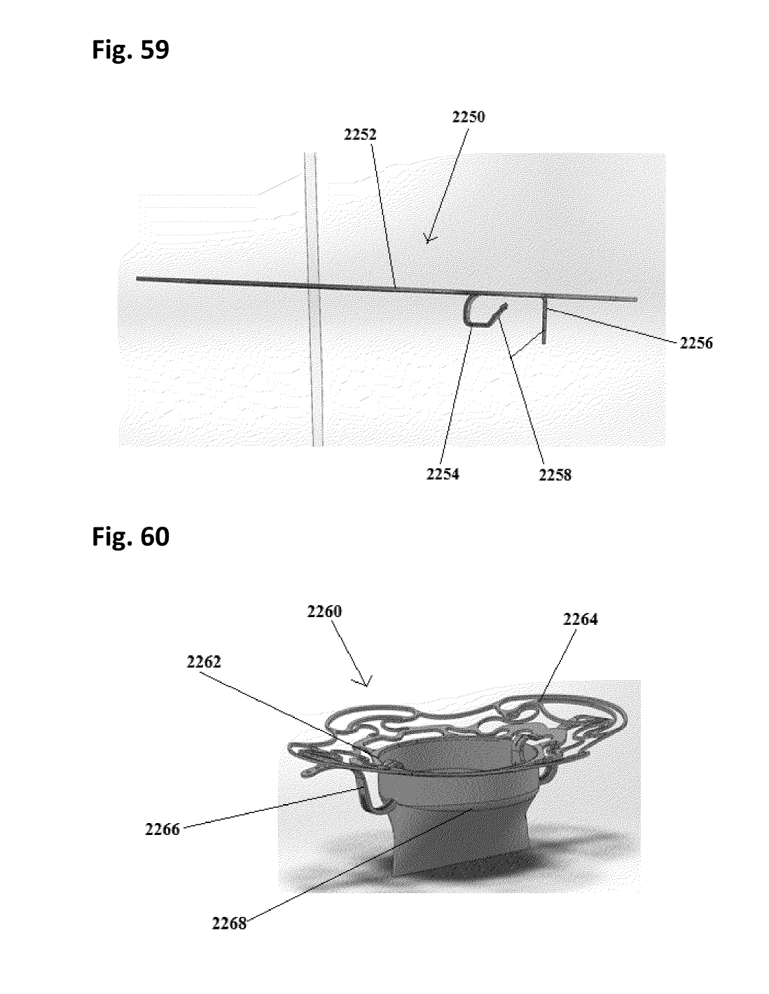

FIG. 59 illustrates a side view of the same embodiment as shown in FIG. 58, in its in situ conformation. It may be seen from this figure that the lateral prong 2256 is straight and angled at about 90 degrees downwards (in relation to the plane of the support ring), while the medial prong 2254 adopts a curved conformation. It should be noted that these two prong conformations are shown for illustrative purposes only, and various other shapes are also included within the scope of the invention.

In another aspect, the present invention also encompasses the use of a skirt-like fabric structure attached to the inner perimeter of the valve support ring. In one preferred embodiment of this aspect, said skirt-like structure is attached to the entire inner circumference of said ring and is disposed such that the body of said skirt passes inferiorly therefrom. Said skirt can be made of a biocompatible fabric, for example PTFE, polyester, Dacron, and can be sutured to the device with a biocompatible surgical suture. In some cases, the desired shape of the fabric skirt will be created by means of constructing said skirt from a thermosetting fabric such as Polyester and applying a source of heat to said material. For example, the thermosetting fabric can be molded to the desired shape using a mandrel at a temperature of approximately 150 degrees Celsius for approximately 15 minutes and attached to the inner surface of the valve support device by means of surgical sutures. FIG. 60 illustrates one preferred embodiment of this aspect, in which a mitral valve support device 2260 comprising a single support ring 2262, a crown-like lateral extension 2264 and two stabilizing wings 2266 is fitted with a full-circumference fabric skirt 2268. As shown in this figure, the lower portions of the fabric skirt may be mutually apposed, while the upper region has a circular outline at its attachment point on the support ring circumference. During the subsequent implantation of a prosthetic valve within the inner space of support ring 2262, the apposed lower portions of skirt 2268 will be separated and caused to form a generally tubular structure that covers the wall of the implanted valve. Since the prosthetic valve wall is entirely covered (on its lateral aspect) by the skirt-like structure of this embodiment, PVL associated with the presence of said prosthetic valve is significantly reduced or eliminated.

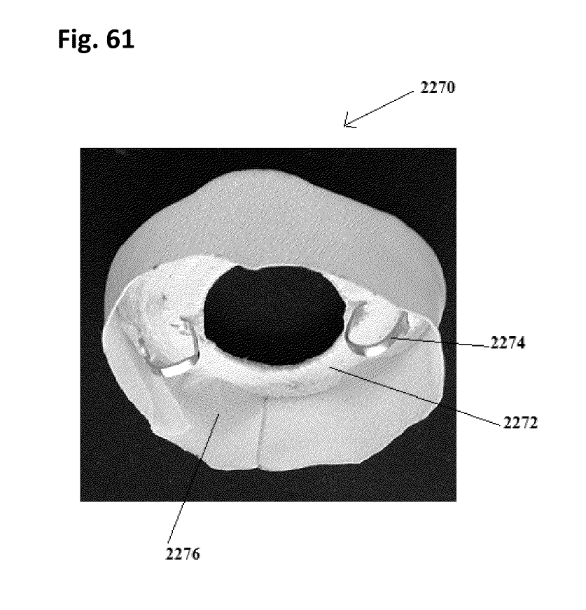

In yet another aspect, the leakage problem has been solved by means of fitting the valve support ring with an inferiorly-disposed sealing drape attached to the outer circumference thereof. While the length of said drape (measured from its point of attachment on the ring to its lower free end) may have any suitable or desired value, in one preferred embodiment, said drape has a length of about 10-20 mm. In one preferred embodiment the drape is constructed from a biocompatible fabric, for example PTFE, polyester, Dacron and is attached to the valve support device by means of surgical sutures. FIG. 61 illustrates one preferred embodiment of this aspect of the present invention, in which a mitral valve support device 2270 comprising a single support ring 2272 and two stabilizing wings 2274 further comprises a fabric drape 2276, wherein said drape is attached to the outer circumference of support ring 2272. As will be noted from this figure, the drape is attached in a continuous manner to the outer edge of the support ring and then passed downwards (for about 20 mm, in the present example), such that when the device is implanted at the mitral annulus, the region that is most susceptible to PVL (i.e. the angle created between the mitral support ring and the atrial wall) is covered by said drape. In this way, PVL (and especially leakage associated with the presence of a subsequently-implanted prosthetic valve) is largely prevented. In other embodiments of this invention the fabric drape may be attached to any other aspect of the ring, instead of, or in addition to the outer circumference. In other embodiments, instead of having one fully circular fabric drape, the seal may be made of two or more fabric drapes, overlapping one another and together forming a fully circular drape. The advantage of this structure is that this allows the device to crimp to a lower crimp diameter, which is important for transcatheter delivery. The drape may also be a partial drape (not fully circular), for example a partial drape only in the area which will be approximated to the anterior (aortic) mitral leaflet, and improve sealing in that area.

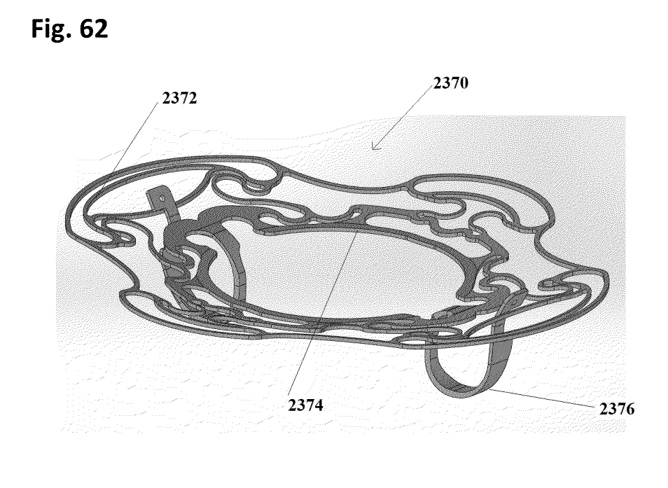

As explained hereinabove, in certain embodiments of the present invention, the annular single-ring valve support device may further comprise one or more lateral extensions as a means for reducing PVL as well as for improving the co-axial positioning of the device. In some embodiments, the extension elements are not continuously connected to the external aspect of the ring along their entire length, but rather are connected to the ring only at discrete singular connection points (for example, connected only at two points, on at the front edge of said element and one at the back edge thereof), without any connection at the central part of the element. In other embodiments, the lateral extensions from a complete crown-like structure around the outer edge of the valve support device. The lateral extensions are deployed on the atrial side of the mitral annulus, above the commissures of the mitral valve, in such a way that they “cover” the space formed by the commissures. FIG. 62 provides an example of a single-ring support device of the present invention 2370 which comprises a crown-like lateral extension structure 2372 encompassing the entire outer aspect of support ring 2374. The valve support device shown in this figure further comprises a pair of anchoring wings 2376.

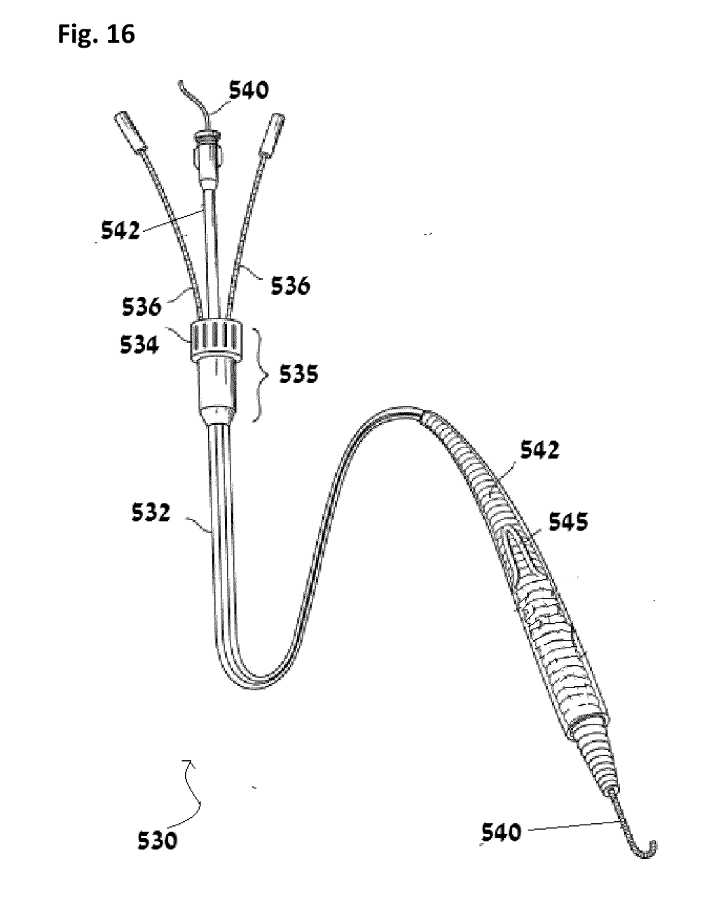

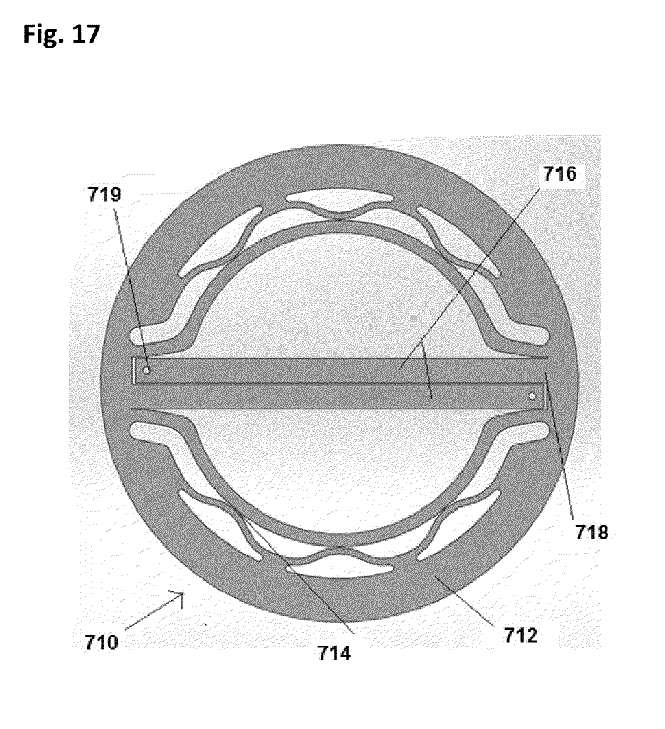

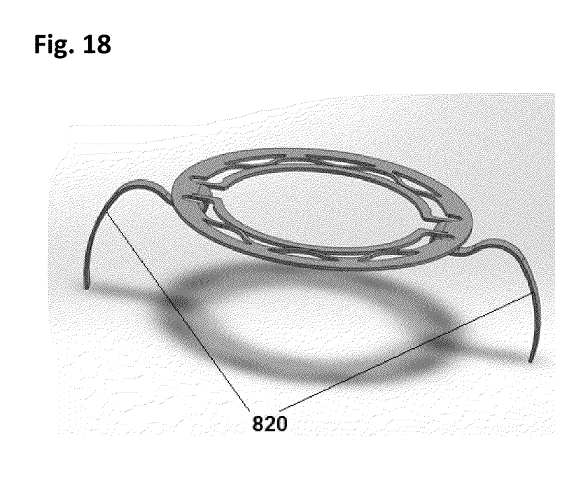

Delivery of the single-ring valve support device of the present invention is accomplished using essentially the same method and delivery device as disclosed in co-owned, co-pending U.S. application Ser. No. 13/224,124, filed on Sep. 1, 2011. Briefly, the method of delivery involves the use of a delivery device 530, as illustrated in perspective view in FIG. 16. The device, as shown in this figure, comprises an actuation portion 535, an actuator, 534, an elongate body 532, and a guidewire lumen 542 which is adapted to be advanced distally over guidewire 540 to advance delivery device 530 to a target location within the subject. The valve support device of the present invention in its collapsed conformation 545 is contained within the lumen of the delivery device, and is connected to coupling members 536. The distal regions of device coupling members 536 are releasably secured to the valve support device during the deployment procedure, but are also adapted to be controllably released from said valve support device, in order to release it from the delivery device. Coupling members 536 can be actuated by actuating their proximal portions external to the patient to control movement of the valve support device.

The elongate body 532 can be, for example without limitation, a catheter, examples of which are well known. Actuation portion 535 can be, for example without limitation, a touhy borst, allowing rotation of actuator 534 to control the axial movement of elongate body 532. Guiding lumen 542 can be, for example without limitation, a corrugated steel reinforced lumen to allow for sufficient flexibility while being advanced through the vasculature. Guiding lumen 542 can also be any other type of suitable guiding lumen.

Following release of the valve support at the desired site, it is allowed to expand in order that it may adopt its working conformation, securing itself against the lateral wall of the cardiac lumen, in the atrium above the mitral valve annulus, in the ventricle below the annulus or within the annulus itself. In some embodiments the support element includes one or more cardiac anchoring elements, such as in the form of anchors, barbs, clips, etc. and/or stabilizing elements (as described hereinabove), that help secure said support element against cardiac tissue, or that are adapted to pierce into cardiac tissue to secure the support element to cardiac tissue. One or more fixation and/or stabilizing elements, if used, can be disposed around the periphery of the support element. They can assume a collapsed, or delivery configuration for delivery of the system, but can deploy to an expanded, or anchoring, configuration, when released from the delivery system. For example, the fixation elements can be an elastic material that self-expands to an anchoring configuration. Alternatively, the fixation elements can be actuated to reconfigure them to a fixation configuration. In some embodiments, however, the one or more fixation elements are not adapted to change configurations. Further details concerning the subsequent stage of deploying and expanding a commercially-available replacement valve (of any type suitable for the procedure in question as determined by the clinician) are disclosed in co-pending U.S. application Ser. No. 13/224,124, filed on Sep. 1, 2011.

Access to the mitral valve or other atrioventricular valve will preferably be accomplished through the patient's vasculature percutaneously (access through the skin). Percutaneous access to a remote vasculature location is well-known in the art. Depending on the point of vascular access, the approach to the mitral valve can be antegrade and require entry into the left atrium by crossing the interatrial septum. Alternatively, approach to the mitral valve may be retrograde where the left ventricle is entered through the aortic valve. Alternatively, the mitral valve can be accessed transapically, a procedure known in the art. Additional details of an exemplary antegrade approach through the interatrial septum and other suitable access approaches can be found in the art, such as in U.S. Pat. No. 7,753,923, filed Aug. 25, 2004, the contents of which are incorporated herein by reference.

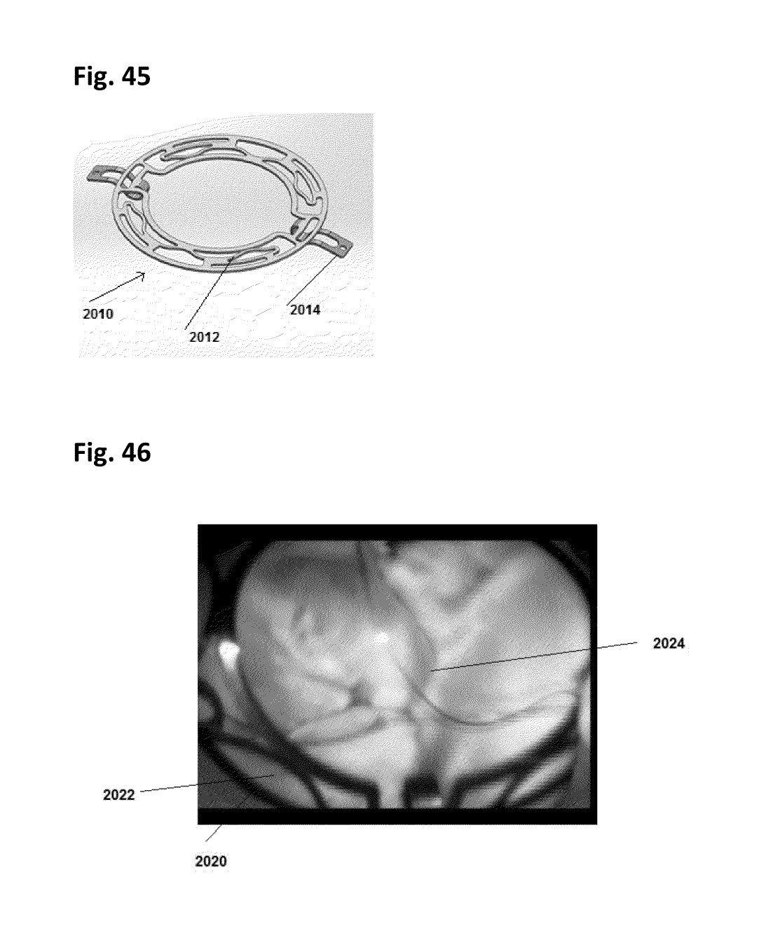

FIGS. 44 and 45 depict two versions of the single-ring valve support device that were developed for use in conjunction with two different classes of prosthetic aortic valve, in order to be able to use said prosthetic valves to replace dysfunctional mitral valves. Thus, FIG. 44 shows, in perspective view, a single-ring support device 2000 for use in conjunction with a self-expanding aortic valve. It may be seen that the device has an outer perimeter 2002 and an inner perimeter 2004, and that a number of linear slots 2006 have been created in the material of the ring between said perimeters. In this way, the inner perimeter of the ring-support has been rendered elastically deformable. The figure also shows that the support device is fitted with two short stabilizing wings 2008 positioned such that they are 180 degrees apart. The support device 2010 shown in FIG. 45 is intended for use with a balloon-expandable aortic valve, and is similar in general structure to the device in FIG. 44, having a series of slots 2012 and two short stabilizing wings 2014. However, the slots shown in the case of this device differ both in terms of the complexity of their shape, and in the fact that they occupy a larger surface area than those shown in the previous figure. These differences result in an inner ring perimeter that has greater elasticity, a feature which is important in relation to the use of this support device in conjunction with balloon-expandable prosthetic aortic valves.

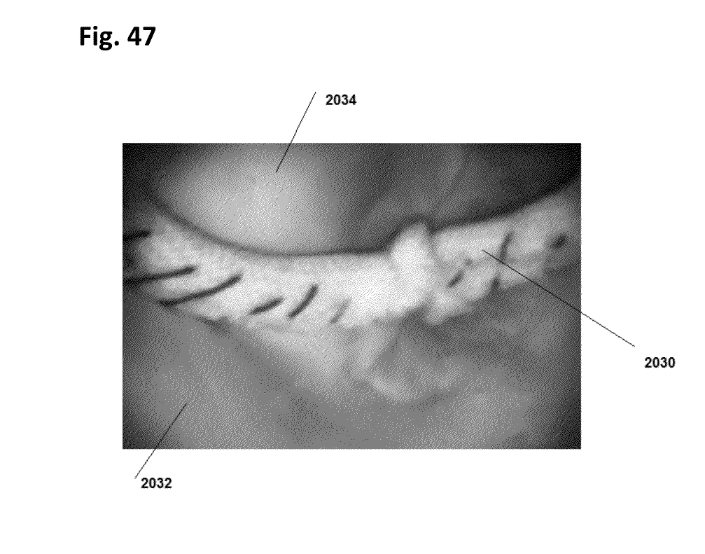

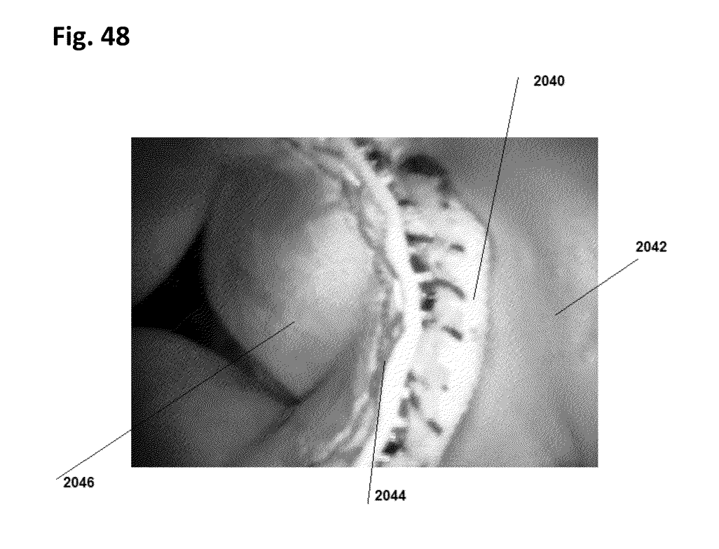

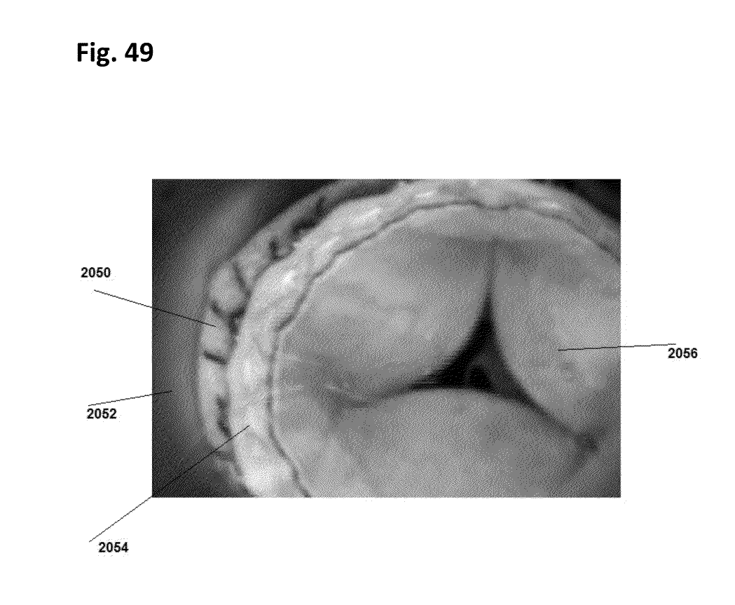

FIGS. 46 to 49 are photographs showing the successful implantation of single-ring valve support devices of the present invention in cadaver hearts. Thus, FIG. 46 shows a single ring support device 2020 implanted in a mitral annulus 2022 position in a cadaveric heart. The heart is connected to a pulsating pump, which provides flow and thus causes the mitral valve to open and close. It is to be noted that the device in this figure is shown without covering fabric. The photograph illustrates that when the support device is located on the annulus, it does not interfere with the closure of the native mitral valve 2024 (shown closed in the photo)—and thus maintains stable hemodynamics and allows for timely and safe deployment of a prosthetic valve within the support device in a two-stage implantation procedure as explained hereinabove. FIG. 47 presents an enlarged view of a similar valve support device 2030, implanted above a mitral annulus 2032, said device being, in this case, covered with a biocompatible fabric. The native mitral valve leaflets 2034 are shown in their closed position. FIG. 48 shows a similar fabric-covered valve support device 2040, positioned above the mitral annulus 2042. In the example shown in this figure, however, a prosthetic aortic valve 2044 has been implanted and expanded within the central space of said support device. The leaflets 2046 of the prosthetic valve are also clearly seen in this figure. FIG. 49 presents an upper view of the same prosthetic valve, having a stent portion 2054 and three leaflets 2056, wherein said valve is firmly held in place by valve support device 2050, which itself is shown implanted above mitral annulus 2052. These photographic pictures of the valve support device of the present invention deployed in cadaveric hearts demonstrate that the stented valve is anchored very firmly to the single ring support device, and is not displaced, even at the high pressures generated by the pulsating valve (pressures greater than 150 mmHg, which are similar to those seen in hypertension).

While the support structures herein are generally described as a support for replacement mitral valves, they can be delivered to a desired location to support other replacement cardiac valves, such as replacement tricuspid valves, replacement pulmonic valves, and replacement aortic valves.

While some embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

1. A prosthetic cardiac valve support device adapted for endovascular delivery to a cardiac valve, comprising a single ring-shaped support element having an inner diameter and an outer diameter, wherein said support element has an outer perimeter that is entirely rigid, wherein said support element is fitted with one or more intra-ventricular and/or intra-atrial stabilizing elements, and wherein said support element has a collapsed delivery configuration and a deployed configuration.

2. The valve support device according to claim 1, wherein the support element in its deployed configuration has the form of a flat annular ring, and wherein the difference (Rd) between the outer radius and the inner radius of said annular ring is in the range of 1-14 mm.

3. The valve support device according to claim 2, wherein the ratio between Rd and the thickness of the single-ring support element is in the range of 10:1 and 20:1.

4. The valve support device according to claim 2, wherein the inner radius of the single-ring support element is in the range of 23-29 mm and the outer radius thereof is in the range of 30-50 mm.

5. The valve support device according to claim 2, wherein the thickness of said support device is in the range of 0.25-0.6 mm.

6. The valve support device according to claim 1, wherein the stabilizing elements are selected from the group consisting of complete ring structures, partial rings, curved arms or wings, elongate arms or wings and levered arms or wings.

7. The valve support device according to claim 1, wherein the inner perimeter of the single-ring support element is able to elastically deform in a radial direction.

8. The valve support device according to claim 1, further comprising means for reducing paravalvular leakage, wherein said means are selected from the group consisting of: lateral edge extensions, one or more tubular sealing elements, one or more barbs, an inferiorly-directed circumferential fabric skirt attached to the inner circumference of the ring-shaped support element and an inferiorly-directed fabric curtain attached to the outer circumference of said support element.

9. The valve support device according to claim 1, wherein the support element is fitted with replacement valve engagement means adapted to securely engage a replacement heart valve.

10. The valve support device according to claim 1, further comprising one or more height-increasing elements attached to the inner circumference of the ring-shaped support element, wherein said height-increasing elements are selected from the group consisting of wire springs and tabs.

11. An endovascular or transapical delivery system for use in the replacement of a mitral valve, comprising:
a) a prosthetic cardiac valve support device comprising a single ring-shaped support element with a collapsed delivery configuration and a deployed configuration, according to any one of the preceding claims; and
b) a replacement heart valve comprising an expandable anchor and a plurality of leaflets;
wherein said replacement heart valve is adapted to be secured to said prosthetic cardiac valve support device.

12. The system according to claim 11, wherein the replacement heart valve is a prosthetic aortic valve.

13. A method for replacing a mitral valve in a patient in need of such treatment, comprising delivering a valve support device to a location near a subject's mitral valve, wherein said valve support device comprises a single ring-shaped support element; expanding the support element from a collapsed configuration to a deployed configuration secured against cardiac tissue above the plane of the mitral valve annulus, below this plane or against the annulus itself, and subsequently delivering a replacement heart valve and securing said replacement heart valve to the valve support device by means of expanding said replacement heart valve from a collapsed delivery configuration to an expanded configuration.

14. The method according to claim 13, wherein said method is used to deliver the valve support device by an endovascular route.

15. The method according to claim 13, wherein said method is used to deliver the valve support device by the transapical route.

16. The method according to claim 13, wherein the valve support device and the replacement heart valve are both delivered by the same route, selected from the group consisting of an endovascular route and the transapical route.

17. The method according to claim 13, wherein the valve support device and the replacement heart valve are delivered by different routes.

18. The method according to claim 13, wherein the replacement heart valve is a prosthetic aortic valve.
